SEQUENCE LISTING (36 sequences)

SEQ ID NO: 1 - protein, 15 aa, Artificial sequence
  Engineered linker
GGGGSGGGGSGGGGS

SEQ ID NO: 2 - protein, 25 aa, Artificial sequence
  Engineered linker
GGGGSGGGGSGGGGSGGGGSGGGGS

SEQ ID NO: 3 - protein, 30 aa, Artificial sequence
  Engineered linker
GGGGSGGGGSGGGGSGGGGSGGGGSGGGGS

SEQ ID NO: 4 - protein, 50 aa, Artificial sequence
  Engineered linker
GGGGSGGGGSGGGGSGGGGSGGGGSGGGGSGGGGSGGGGSGGGGSGGGGS

SEQ ID NO: 5 - protein, 36 aa, Artificial sequence
  Engineered linker
GGSSGSGSGSTGTSSSGTGTSAGTTGTSASTSGSGS

SEQ ID NO: 6 - protein, 72 aa, Artificial sequence
  Engineered linker
GGSSGSGSGSTGTSSSGTGTSAGTTGTSASTSGSGSGGSSGSGSGSTGTSSSGTGTSAGTTGTSASTSGSGS

SEQ ID NO: 7 - protein, 454 aa, Artificial sequence
  mAb158 chimeric heavy chain
EVKLMESGGGLVQPGGSRKLSCAASGFTFSSFGMHWVRQAPEKGLEWVAYISSGSSTIYYGDTVKGRFTISRDNPKNTLFLQMTSLRSEDTAMYYCAREGGYYYGRSYYTMDYWGQGTSVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 219 aa, Artificial sequence
  mAb158 chimeric light chain
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 241 aa, Artificial sequence
  Engineered 8D3 scFv
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 10 - protein, 730 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGSSGSGSGSTGTSSSGTGTSAGTTGTSASTSGSGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 729 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 787 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGSSGSGSGSTGTSSSGTGTSAGTTGTSASTSGSGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGSSGSGSGSTGTSSSGTGTSAGTTGTSASTSGSGSGGSSGSGSGSTGTSSSGTGTSAGTTGTSASTSGSGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - protein, 724 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 14 - protein, 744 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 15 - protein, 488 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 675 aa, Mus musculus
CKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 17 - protein, 214 aa, Rattus norvegicus
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 18 - protein, 231 aa, Rattus norvegicus
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGHHHHHHHHHH

SEQ ID NO: 19 - protein, 442 aa, Rattus norvegicus
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 20 - protein, 42 aa, Homo sapiens [sequence omitted]

SEQ ID NO: 21 - protein, 241 aa, Artificial sequence
  Engineered 8D3 scFv reversed
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 22 - protein, 10 aa, Artificial sequence
  Engineered linker
GGGGSGGGGS

SEQ ID NO: 23 - protein, 20 aa, Artificial sequence
  Engineered linker
GGGGSGGGGSGGGGSGGGGS

SEQ ID NO: 24 - protein, 699 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - protein, 709 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 26 - protein, 719 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 27 - protein, 714 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 28 - protein, 719 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 29 - protein, 724 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 30 - protein, 714 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 31 - protein, 719 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 724 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 33 - protein, 454 aa, Artificial sequence
  mAb158 chimeric heavy chain
EVKLMESGGGLVQPGGSRKLSCAASGFTFSSFGMHWVRQAPEKGLEWVAYISSGSSTIYYGDTVKGRFTISRDNPKNTLFLQMTSLRSEDTAMYYCAREGGYYYGRSYYTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 219 aa, Artificial sequence
  mAb158 chimeric light chain
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 238 aa, Artificial sequence
  Engineered 15G11 scFv
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSGGGGSGGGGSGGGGSDIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 36 - protein, 706 aa, Artificial sequence
  Engineered single chain component
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSGGGGSGGGGSGGGGSDIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0141027 A1
(43) Pub. Date: May 2, 2024

(54) BISPECIFIC BINDING MOLECULE

(71) Applicant: BioArctic AB, Stockholm (SE)

(72) Inventors: Ronny Falk, Sundbyberg (SE); Per-Ola Freskgård, Norrköping (SE); Ken Honek, Solna (SE); Lisa Sandersjöö, Stockholm (SE)

(21) Appl. No.: 18/569,061

(22) PCT Filed: Jun. 10, 2022
(86) PCT No.: PCT/EP2022/065915

§ 371 Date: Dec. 11, 2023

(30) Foreign Application Priority Data
Jun. 11, 2021 (EP) 21179103.3

(51) Int. Cl. C07K 16/18

(52) U.S. Cl. CPC C07K 16/18; A61K 2039/505; C07K 2317/31; C07K 2317/622

(57) ABSTRACT

A bispecific binding molecule is provided, which comprises two identical antibody heavy chains and a single chain component which is a polypeptide chain comprising two identical antibody light chains linked to a single chain binding module with affinity for a target which mediates transport of the bispecific binding molecule across the blood-brain barrier (BBB). Also provided are therapeutic, prophylactic, prognostic and diagnostic uses of the bispecific binding molecule.

### TECHNICAL FIELD

The present disclosure relates to a bispecific binding molecule comprising two identical antibody heavy chains and a single chain component which is a polypeptide chain comprising two identical antibody light chains linked to a single chain binding module with affinity for a target which mediates transport of the bispecific binding molecule across the blood-brain barrier (BBB). The antibody heavy and light chains are derived from a monoclonal antibody with affinity for a target present in the brain of a mammal.

The disclosure also relates to therapeutic, prophylactic, prognostic and diagnostic uses of the bispecific binding molecule.

### BACKGROUND

Treatment modalities for brain and neurological diseases are extremely limited due to the impermeability of the blood vessels of the brain to most substances carried in the bloodstream (Freskgard and Urich (2017), Neuropharmacology 120:38-55; Stanimirovic et al (2018), BioDrugs 32:547-559). The small blood vessels (capillaries) of the brain, referred to collectively as the blood-brain barrier (BBB), are unique when compared to the blood vessels found in the periphery of the body. Tight apposition of BBB endothelial cells (EC) to neural cells, such as astrocytes, pericytes and neurons, induces phenotypic features that contribute to the observed impermeability. Tight junctions between ECs in the BBB limit paracellular transport, while the lack of passive pinocytotic vesicles and fenestrae limit non-specific transcellular transport. These factors combine to restrict molecular flux from the blood to the brain in general to molecules that are less than 500 Da in size and lipophilic. Thus, the otherwise promising prospect of using the large mass transfer surface area (over 20 m² from 600 km of capillaries in a human brain) of the blood stream as a delivery vehicle is made largely infeasible, except in those circumstances where a drug with the desired pharmacological properties fortuitously possesses size and lipophilicity attributes which allow it to pass through the BBB. Because of such restrictions, it has been estimated that more than 98% of all small molecule pharmaceuticals and nearly 100% of the emerging class of protein and gene therapeutics do not cross the BBB.

WO91/03259 proposes a principle for transporting a neuropharmaceutical agent across the BBB, which involves conjugating the agent to an antibody which is reactive with the transferrin receptor. According to this disclosure, binding of the conjugate to the transferrin receptor leads to active transport of the conjugate across the BBB. Later work developed this basic concept further, e.g. proposing other receptors that could be useful for BBB transport, as alternatives to the transferrin receptor.

WO2012/075037 discloses a bispecific antibody having one Fab part which is specific for a BBB receptor and mediates transport, and one Fab part which is specific for a therapeutic target in the brain. FIG. 3A of WO2012/075037 illustrates a representative embodiment of this teaching.

WO2014/033074 discloses a bispecific binding molecule comprising a standard, monospecific and bivalent antibody directed against a therapeutic target in the brain, and a binding domain which is specific for a BBB receptor. The binding domain is coupled to the C-terminus of one of the heavy chains of the antibody. FIG. 1B of WO2014/033074 illustrates a representative embodiment of this teaching. Furthermore, the document discloses that monovalent binding to the BBB receptor leads to a more efficient BBB transport than bivalent binding. It is believed that the candidate biopharmaceutical product denoted R07126209 from Roche employs the design described in WO2014/033074. R07126209 is undergoing clinical trials with the identifiers NCT04023994 and NCT04639050 on clinicaltrials.gov.

WO2018/011353 discloses another alternative construct, in which a standard, monospecific and bivalent antibody directed against a therapeutic target in the brain was equipped with a plurality of BBB receptor binding elements, that are nevertheless arranged such that the desired monovalent binding to the BBB receptor is possible. FIG. 1A of WO2018/011353 illustrates a representative embodiment of this teaching.

Despite the existence of various experimental formats for the provision of antibody and antibody-derived biopharmaceuticals with the ability to cross the BBB via active transport mediated by the transferrin receptor and other receptors, all of these suffer from one or more drawbacks, and there remains a need in the art for novel biopharmaceuticals that can cross the BBB, for the further development of highly functional therapeutic, prophylactic, diagnostic and prognostic tools for detecting and treating diseases of the brain and central nervous system, for example Alzheimer's disease and other neurodegenerative diseases.

### DESCRIPTION OF THE INVENTION

It is an object of the invention to provide a design format which enables antibody-based therapeutics to reach the brain.

Another object of the invention is to provide a format for a bispecific binding molecule with affinities for a brain target and for a BBB transport mediator.

Another object of the invention is to provide a symmetric format which is easily expressed and assembled, while at the same time providing a monovalent binding to a BBB transport mediator.

Another object of the invention is to provide a symmetric format which prevents the formation of any product that may lead to bivalent interaction with the BBB transport mediator.

Another object of the invention is to produce a symmetric bispecific binding molecule ready for use from only two different continuous polypeptide chains.

One or more of these objects, and other objects that are apparent to the skilled person from reading the entire disclosure, are met by the various aspects disclosed.

Thus, in a first aspect, the present disclosure provides a bispecific binding molecule, comprising the following three polypeptide chains:
(A) two identical antibody heavy chains (HC) derived from a monoclonal antibody with affinity for a first target present in the brain of a mammal; and
(B) a single chain component, which comprises the following five elements in a continuous polypeptide chain:
   i) two identical antibody light chains (LC) derived from said monoclonal antibody with affinity for said first target;
   ii) one single chain binding module (scBM) with affinity for a second target which mediates transport of the bispecific binding molecule through the blood-brain barrier; and
   iii) two amino acid linkers L1 and L2, wherein said light chains (LC) and said single chain binding module (scBM) are separated by said linkers L1 and L2, thus forming a sequence from the N terminus to the C terminus selected from the group consisting of
      [LC-L1-scBM-L2-LC],
      [LC-L1-LC-L2-scBM] and
      [scBM-L1-LC-L2-LC].

A bispecific binding molecule as defined above will be formed through the association of each light chain LC element in the single chain component with each heavy chain HC, so that the bispecific binding molecule adopts a standard antibody configuration that essentially recreates the monoclonal antibody with affinity for a first target from which the heavy (HC) and light (LC) chains are derived, having the single chain binding module scBM coupled thereto via the linkers L1 and L2 (see FIGS. 1 and 2 for illustrations of two different embodiments of the bispecific binding molecule).

Without wishing to be bound by theory, and as non-limiting examples, the bispecific binding molecule of the disclosure offers the following advantages over existing formats for transport of therapeutic antibodies across the BBB.

The constructs disclosed in WO2012/075037 and WO2014/033074 are both asymmetric, requiring the expression of different heavy chain elements that must then be assembled by use of e.g. knob-into-hole technology. This may lead to inefficiencies in production, potential for chain imbalance problems and the associated generation of unwanted side products (e.g. homodimeric knob-knob and hole-hole side products; see Kuglstatter et al (2017), Protein Eng Des Sel 30:649-656). For any antibody heterodimerization technology, a low content or even absence of homodimeric side product impurity is desired. This applies in cases where undesired homodimers have biophysical properties that are similar to those of the desired product, making them difficult to separate using production scale purification processes, as well as in cases where undesired homodimers retain biologic activity, such as an affinity for a BBB transport mediator. This is especially important when the homodimeric side product has the potential to induce undesirable biological activities, such as activities which lead to dimerization or multimerization of the BBB transport mediator, in turn compromising the function of the molecule. The bispecific binding molecule of the present disclosure, on the other hand, comprises only one type of heavy chain and is, in this sense, a symmetric construct.

The construct disclosed in WO2018/011353 does not suffer from the drawbacks associated with heavy chain asymmetry, but may instead fail to provide the necessary monovalent binding to the mediator of BBB transport because it comprises two separate BBB binding moieties. Even though the inventors in WO2018/011353 disclose that the bivalent construct nevertheless confers monovalent binding in certain situations, there remains an uncertainty regarding the general applicability of the principle in WO2018/011353. With the bispecific binding molecule of the present disclosure, this uncertainty is removed, and true monovalent binding to the mediator of BBB transport is achieved.

### Overall Architecture of the Single Chain Component

According to the disclosure, the single chain component comprises five elements in a continuous polypeptide chain. One of the five elements is a single chain binding module (scBM) which has an affinity of a target which mediates transport through the BBB. Two of the five elements are identical antibody light chains (LC), which combine with the two identical heavy chains (HC) to form an antibody part of the bispecific binding molecule. As appreciated by a person of skill in the art, the scBM and LC elements in the single chain component must be separated in order that each of them may serve their purpose of either associating with a heavy chain (HC) (in the case of the light chain elements) or providing the bispecific binding molecule with a binding affinity for the BBB target (in the case of the scBM). For this purpose, these three elements are separated by the two remaining elements of the single chain component, i.e. the two amino acid linkers L1 and L2. In this regard, it is contemplated that any order of the elements in the single chain component is possible, as long as the linkers separate the three binding elements. In other words, the three possible sequences of elements in the single chain component of the bispecific binding molecule are the following, from the N terminus to the C terminus:
[LC-L1-scBM-L2-LC]
[LC-L1-LC-L2-scBM], and
[scBM-L1-LC-L2-LC].

In one embodiment, the sequence of elements is selected from [LC-L1-scBM-L2-LC] and [LC-L1-LC-L2-scBM]. In a specific embodiment, the sequence of elements is [LC-L1-scBM-L2-LC]. In another specific embodiment, the sequence of elements is [LC-L1-LC-L2-scBM].

### Antibody Heavy and Light Chains

In the bispecific binding molecule of the disclosure, the antibody heavy chain and antibody light chain elements, i.e. HC and LC, are each derived from a monoclonal antibody with affinity for a first target present in the brain of a mammal. As used herein in this specific context, “derived from” means that the amino acid sequences of each of HC and LC are essentially unchanged in comparison to the sequences of the “parent” monoclonal antibody from which the HC and LC are derived. In other words, the bispecific binding molecule of the disclosure incorporates the heavy and light chains of the monoclonal antibody as HC and LC, respectively.

In the bispecific binding molecule of the disclosure, the two antibody heavy chains (HC) and the two light chain elements (LC) in the single chain component combine to form a traditional antibody structure, with the scBM attached thereto via the L1 and L2 linkers. As such, the bispecific binding molecule will comprise two VH-VL pairs from each pair of HC and LC, and have the ability to bind to the first target via complementarity determining regions (CDRs), in the same or essentially the same way as the monoclonal antibody from which they are derived.

Through its combined HC and LC elements, the bispecific binding molecule of the disclosure has affinity for a first target present in the brain of a mammal. This target is typically associated with a disease, in such a way that binding, blocking, activating or otherwise interacting with the target is desired or beneficial in the context of treating or preventing such disease. However, the particular benefits of the bispecific binding molecule according to the disclosure come primarily from the enhanced capacity of the target binding activity to reach its intended destination in the brain, and not from the fact that it binds to any particular first target. As such, the specific nature of the first target is not limited but can be any target in the brain with which it may be of interest to interact.

Nevertheless, by way of illustration, said first target may in certain embodiments be selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), beta-secretase 1 (BACE1), superoxide dismutase (SOD), huntingtin, transthyretin, P-secretase 1, epidermal growth factor, epidermal growth factor receptor 2, Tau, phosphorylated Tau or fragments thereof, apolipoprotein E4, CD20, prion protein, leucine rich repeat kinase 2, parkin, presenilin 2, gamma secretase, death receptor 6, amyloid-β precursor protein, p75 neurotrophin receptor, neuregulin and caspase 6.

In a more specific embodiment, the first target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), Tau, phosphorylated Tau or fragments thereof and apolipoprotein E4.

In a particularly specific embodiment, the first target is selected from the group consisting of amyloid-β peptide or derivatives or fragments thereof, alpha-synuclein or derivatives or fragments thereof and TAR DNA-binding protein 43 (TDP-43) or derivatives or fragments thereof.

Any monoclonal antibody which binds to any one or more of the targets listed above and present in the brain of a mammal is contemplated to be useful as a source of HC and LC elements in the bispecific binding molecule of the disclosure. The skilled person within the field of biopharmaceutical research is aware of a number of monoclonal antibodies with affinity for such targets.

In one embodiment, the monoclonal antibody from which the HC and LC elements are derived is of IgG class. In a more specific embodiment, the antibody or antigen-binding fragment thereof is of a sub-class selected from IgG1, IgG2 and IgG4, for example selected from IgG1 and IgG4. The desired sub-class used is for example dependent on the required function of the monoclonal antibody. In a more specific embodiment, the antibody is of the sub-class IgG1. An IgG1 antibody is especially preferred when e.g. effective antibody-dependent cell-mediated cytotoxicity (ADCC), antibody-dependent cellular phagocytosis (ADCP) or complement-dependent cytotoxicity (CDC) is desired.

In one embodiment, the monoclonal antibody from which the HC and LC elements are derived is selected from the group consisting of human antibodies, humanized antibodies and antibodies that have been mutated to reduce effector function, prolong plasma half-life or reduce the antigenicity thereof in humans.

Non-limiting examples of specific monoclonal antibodies from which HC and LC elements may be derived are known antibodies directed against various forms of amyloid-β, such as selected from the group consisting of lecanemab, gantenerumab, aducanumab, donanemab, PBD-C06 and KHK6640. Other examples of monoclonal antibodies from which HC and LC elements may be derived are antibodies directed against alpha-synuclein, for example ABBV0805.

In the following Examples, the concept of the bispecific binding molecule of the disclosure is tested and found to work in the intended way. Examples 10, 14 and 15 in particular show different bispecific binding molecules as defined herein in mouse studies in vivo. In order to serve as proper proof-of-concept constructs in mice, the bispecific binding molecules tested in the Examples have antibody heavy and light chain variable domains from a mouse monoclonal antibody with affinity for amyloid p protofibrils, mAb158, rather than from any of the human, humanized or chimeric antibodies that are discussed above and are intended for use in humans. The mouse antibody mAb158 is the mouse progenitor to the humanized monoclonal antibody lecanemab (a.k.a. BAN2401; see WO2007/108756).

### Single Chain Binding Module

In one embodiment, the single chain binding module (scBM) element of the single chain component is of antibody origin. The scBM may for example be selected from known single chain formats, such as from the group consisting of scFv, scFab, VHH and VNAR. In one embodiment, the scBM is selected from the group consisting of scFv and scFab. In a more specific embodiment, the scBM is a scFv. In another specific embodiment, the scBM is a scFab.

In another embodiment, the scBM, element of the single chain component is not of antibody origin. In this embodiment, the scBM may for example be selected from known non-antibody scaffolds, such as from the group consisting of monobodies (Adnectin® molecules), protein Z variants (Affibody® molecules), lipocalins (Anticalin® proteins), bicyclic peptides, ankyrin repeat proteins (DARPin® molecules), fynomers and Kunitz domains.

The single chain binding module (scBM) provided in the single chain component of the bispecific binding molecule of the disclosure has affinity for a second target, which mediates transport of the bispecific binding molecule through the BBB when administered to a subject. This second target may for example be a receptor or other ligand found on the surfaces of the endothelial cells of the BBB. The skilled person is aware of numerous different targets that have been tested for the purpose of BBB transport, or “brain shuttling”, and may select a suitable single chain binding module based on its affinity for such a target.

In one embodiment, said second target is selected from the group consisting of transferrin receptor 1 (TfR1), insulin receptor (InsR), insulin-like growth factor 1 receptor (IGF-1R), low density lipoprotein receptor-related protein 8 (Lrp8), low density lipoprotein receptor-related protein 1 (Lrp1), CD98, transmembrane protein 50A (TMEM50A), glucose transporter 1 (Glut1), basigin (BSG) and heparin-binding epidermal growth factor-like growth factor.

In a more specific embodiment, said second target is selected from the group consisting of transferrin receptor 1 (TfR1), insulin receptor (InsR), insulin-like growth factor 1 receptor (IGF-1R) and low density lipoprotein receptor-related protein 8 (Lrp8).

In an even more specific embodiment, said second target is transferrin receptor 1 (TfR1).

In the proof-of-concept studies reported in the following Examples, the bispecific binding molecules tested employ a scFv domain with affinity for mouse transferrin receptor 1 (mTfR1) as scBM. The scFv domain is constructed from the VH and VL domains of the monoclonal mouse anti-mTfR1 antibody 8D3 (Kissel et al (1998), Histochem Cell Biol 110:63-72).

### Linkers L1 and L2

With respect to the design of linkers L1 and L2, the skilled person is aware that the construction of a fusion protein, such as the single chain component of the bispecific binding molecule, often involves the use of linkers between the functional moieties to be fused, and that there are different kinds of linkers with different properties, such as flexible amino acid linkers, rigid amino acid linkers and cleavable amino acid linkers. As disclosed above, the single chain component in the bispecific binding molecule according to the disclosure comprises two linkers L1 and L2. In one embodiment, one or both of L1 and L2 is/are suitably selected from flexible amino acid linkers, rigid amino acid linkers and cleavable amino acid linkers. In one embodiment, at least one of L1 and L2 is a flexible amino acid linker. In another embodiment, both L1 and L2 are flexible amino acid linkers. As known by a person of skill in the art, flexible linkers are often used when the joined domains or elements require a certain degree of movement or interaction, and may be particularly useful in some embodiments of the bispecific binding molecule of this disclosure. Flexible linkers are generally composed of small, non-polar (for example G or A) or polar (for example S or T) amino acids. Some flexible linkers primarily consist of stretches of G and S residues, for example (GGGGS)ₚ. Adjusting the copy number “p” allows for optimization of a linker in order to achieve appropriate separation between the functional moieties or to maintain necessary inter-moiety interaction. Apart from G and S linkers, other flexible linkers are known in the art, such as G and S linkers containing additional amino acid residues, such as T and A, to maintain flexibility, and/or polar amino acid residues to improve solubility.

In one embodiment of the bispecific binding molecule of the disclosure, at least one of the linkers L1 and L2 is a flexible linker comprising glycine (G), serine (S), alanine (A) and/or threonine (T) residues. In another embodiment, both linkers L1 and L2 is such a flexible linker.

In one embodiment of the bispecific binding molecule of the disclosure, at least one of the linkers L1 and L2 has a general formula selected from (GₙSₘ)ₚ and (SₙGₘ)ₚ, wherein, independently, n=1-7, m=0-7, n+m s 8 and p=1-10. In one embodiment, n=1-5. In one embodiment, m=0-5. In one embodiment, p=3-10. In a more specific embodiment, n=4, m=1 and p=1-4. In one embodiment, at least one of the linkers L1 and L2 is selected from the group consisting of (G₄S)₃ (SEQ ID NO:1), (G₄S)₅ (SEQ ID NO:2), (G₄S)₆ (SEQ ID NO:3) and (G₄S)₁₀ (SEQ ID NO:4). In one particular embodiment, at least one of L1 and L2 is (G₄S)₃. In another embodiment, at least one of L1 and L2 is (G₄S)₅.

In one embodiment of the bispecific binding molecule of the disclosure, at least one of the linkers L1 and L2 is a flexible linker comprising G, S, T and A residues. In one such embodiment, at least one of L1 and L2 has the amino acid sequence SEQ ID NO:5. In another embodiment, at least one of L1 and L2 has the amino acid sequence SEQ ID NO:6.

In one embodiment of the bispecific binding molecule of the disclosure, L1 and L2 are identical. In another embodiment, L1 and L2 are different.

In one embodiment, L1 and L2 have the same length, i.e. have the same number of amino acid residues. In another embodiment, L1 and L2 are of different lengths. In such embodiments, L1 may be longer than L2, or vice versa.

In one embodiment, L1 and/or L2 is/are between 10 and 50 amino acid residues long, such as between 10 and 30 amino acid residues long, such as between 15 and 25 amino acid residues long, or, alternatively between 10 and 20 amino acid residues long.

### Affinity for a Target

As used herein, the terms “specific binding to X”, “selective binding to X” and “affinity for X”, wherein X is a target (e.g. an antigen or an epitope), refer to a property of a binding molecule, such as an antibody or antigen-binding fragment thereof, which may be tested for example by ELISA, by surface plasmon resonance (SPR), by Kinetic Exclusion Assay (KinExA®) or by bio-layer interferometry (BLI). The skilled person is aware of these methods and others.

For example, binding affinity for a target, antigen or epitope X may be tested in an experiment in which a binding molecule to be tested is captured on ELISA plates coated with X or a molecule comprising the epitope X, and a biotinylated detector antibody is added, followed by streptavidin-conjugated horse radish peroxidase (HRP). Alternatively, said detector antibody may be directly conjugated with HRP. Tetramethylbenzidine (TMB) substrate is added and the absorbance at 450 nm is measured using an ELISA multi-well plate reader. The skilled person may then interpret the results obtained by such experiments to establish at least a qualitative measure of the binding affinity for X of the binding molecule. If a quantitative measure is desired, for example to determine the EC50 value (the half maximal effective concentration) for the interaction, ELISA may also be used. The response of the binding molecule against a dilution series of X may be measured using ELISA as described above. The skilled person may then interpret the results obtained by such experiments and EC50 values may be calculated from the results, using for example GraphPad Prism v.9 and non-linear regression.

As used herein, the term “EC50” refers to the half maximal effective concentration of binding molecule which induces a response halfway between the baseline and maximum after a specified exposure time.

Additionally, inhibition ELISA may be used to obtain a quantitative measure of interaction by determination of the “IC50” (the half maximal inhibitory concentration). In an inhibition ELISA, the concentration of target X in a fluid sample is measured by detecting interference in an expected signal output. In principle, a known target or epitope-bearing substance is used to coat a multi-well plate. In parallel, a binding molecule with putative affinity for the target is added and incubated with a solution containing target at varied concentrations. Following standard blocking and washing steps, samples containing the mixture of said binding molecule and the target are added to the well. Labeled detection antibody with affinity for the binding molecule is then applied for detection using relevant substrates (for example TMB). In principle, if there is a high concentration of target in the fluid sample, a significant reduction in signal output will be observed. In contrast, if there is very little target in the fluid sample, there will be very little reduction in the expected signal output. The skilled person appreciates that the signal output is also dependent on the affinity of the binding molecule for said target.

As used herein, the term “IC50” refers to the half maximal inhibitory concentration of a binding molecule which induces a response halfway between the baseline and maximum inhibition after a specified exposure time. Herein, a lower IC50 value indicates that a lower concentration of target is required to interfere with the binding of the detection antibody to the known target coated on the plate, as compared to a higher IC50 value. Thus, a lower IC50 value typically corresponds to a higher affinity.

The binding affinity of a binding molecule may also be tested by surface plasmon resonance (SPR). For example, the affinity may be tested in an experiment in which target or epitope X is immobilized on a sensor chip of the instrument, and the sample containing the binding molecule to be tested is passed over the chip. Alternatively, the binding molecule to be tested may be immobilized on a sensor chip of the instrument, and a sample containing X is passed over the chip. The skilled person may then interpret the results obtained by such experiments to establish at least a qualitative measure of the binding affinity for X of the binding molecule. If a quantitative measure is desired, for example to determine a K_{D} value for the interaction, SPR may also be used. Binding values may for example be defined in a Biacore (Cytiva) or ProteOn XPR 36 (Bio-Rad) instrument. The target or epitope is suitably immobilized on a sensor chip of the instrument, and samples of the binding molecule whose affinity is to be determined are prepared by serial dilution and injected. K_{D} values may then be calculated from the results using for example the 1:1 Langmuir binding model of the Biacore Insight Evaluation Software 2.0 or other suitable software, typically provided by the instrument manufacturer.

The binding affinity may also be measured by bio-layer interferometry (BLI), a label-free technology for measuring biomolecular interactions within the interactome. It is an optical analytical technique that analyzes the interference pattern of white light reflected from two surfaces: a layer of immobilized protein on the biosensor tip, and an internal reference layer. The binding between a ligand (target or epitope X) immobilized on the biosensor tip surface and an analyte (such as a binding molecule with a putative affinity for X) in solution produces an increase in optical thickness at the biosensor tip resulting in a wavelength shift, Δλ, which is a direct measure of the change in thickness of the biological layer. Interactions are measured in real time, providing the ability to monitor binding specificity, rates of association and dissociation, or concentration, with precision and accuracy.

The skilled person is aware of the above mentioned and other methods for measuring the affinity of a binding molecule for a target or epitope X, either qualitatively or quantitatively or both.

### Pharmaceutical Compositions

In a second aspect, the disclosure provides a pharmaceutical composition comprising a bispecific binding molecule as described herein and at least one pharmaceutically acceptable excipient or carrier.

Techniques for formulating polypeptides such as antibodies and their derivatives for human therapeutic use are well known in the art and are reviewed, for example, in Wang et al (2007), J Pharm Sci, 96:1-26, the contents of which are incorporated herein in their entirety.

Pharmaceutically acceptable excipients that may be used to formulate the compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene block polymers, polyethylene glycol and wool fat.

In certain embodiments, the pharmaceutical compositions are formulated for administration to a subject via any suitable route of administration including but not limited to intramuscular, intravenous, intradermal, intraperitoneal injection, subcutaneous, epidural, nasal, oral, rectal, topical, inhalational, buccal (e.g., sublingual), and transdermal administration. In preferred embodiments, the composition is formulated for intravenous or subcutaneous administration.

### Methods of Prevention, Treatment, Diagnosis, Prognosis and Detection

The bispecific binding molecule according to the present disclosure may be useful as a therapeutic, prophylactic, diagnostic and/or prognostic agent.

Hence, in a further aspect of the disclosure, there is provided a bispecific binding molecule according to the first aspect, or a pharmaceutical composition according to the second aspect, for use as a medicament.

In yet another aspect of the disclosure, there is provided a bispecific binding molecule according to the first aspect, or a pharmaceutical composition according to the second aspect, for use as a diagnostic agent.

In yet another aspect of the disclosure, there is provided a bispecific binding molecule according to the first aspect, or a pharmaceutical composition according to the second aspect, for use as a prognostic agent.

Also provided are methods of preventing, treating or diagnosing disease or assessing disease prognosis, wherein a bispecific binding molecule as disclosed herein is administered to a subject in need thereof, typically a human subject.

Also provided is the use of the disclosed bispecific binding molecule for the manufacture of compositions (such as medicaments) for use in the prevention, treatment, diagnosis and/or prognosis of any one of the listed diseases.

Thus, in one embodiment, the bispecific binding molecule, or pharmaceutical composition comprising it, is useful in the treatment, prevention, diagnosis and/or prognosis of a neurodegenerative disorder, for example a disorder selected from Alzheimer's disease and other disorders associated with Aβ protein aggregation, traumatic brain injury (TBI), Lewy body dementia (LBD), Down's syndrome (DS), amyotrophic lateral sclerosis (ALS), frontotemporal dementia, tauopathy, systemic amyloidosis, atherosclerosis, Parkinson's disease (PD), Parkinson's disease dementia (PDD), the Lewy body variant of Alzheimer's disease, multiple system atrophy, psychosis, schizophrenia, Creutzfeldt-Jakob disease, Huntington's disease, and familial amyloid neuropathy.

In a more specific embodiment, said disorder is selected from Alzheimer's disease and other disorders associated with Aβ protein aggregation, Lewy body dementia (LBD), Down's syndrome (DS), amyotrophic lateral sclerosis (ALS), frontotemporal dementia, tauopathy, Parkinson's disease (PD), Parkinson's disease dementia (PDD) and the Lewy body variant of Alzheimer's disease.

In an even more specific embodiment, said disorder is selected from Alzheimer's disease and other disorders associated with Aβ protein aggregation, Lewy body dementia (LBD), amyotrophic lateral sclerosis (ALS) and Parkinson's disease (PD), in particular Alzheimer's disease.

In an alternative embodiment, the bispecific binding molecule, or pharmaceutical composition comprising it, is useful in the treatment, prevention, diagnosis and/or prognosis of another disorder, for example a disorder selected from brain cancer, multiple sclerosis and lysosomal storage diseases.

In another aspect, there is provided a method of treatment, prevention, diagnosis and/or prognosis of a disorder as listed above, said method comprising administering to said mammal an amount, such as a therapeutically effective amount, of a bispecific binding molecule, or pharmaceutical composition comprising it.

### INCORPORATION BY REFERENCE

Various publications are cited in the present application, each of which is incorporated by reference herein in its entirety.

### BRIEF DESCRIPTION OF THE FIGURES

FIG. 1 is a schematic overview of one alternative embodiment of the bispecific binding molecule of the disclosure, wherein the elements of the single chain component are in the sequence [LC-L1-scBM-L2-LC]. Each heavy chain HC consists of VH, CH1, CH2 and CH3 immunoglobulin domains. The single chain component consists of a first light chain LC, which in turn consists of VL and CL immunoglobulin domains; a first linker L1; a single chain binding module in the form of an scFv; a second linker L2; and a second LC which is identical to the first.

FIG. 2 is a schematic overview of another alternative embodiment of the bispecific binding molecule of the disclosure, wherein the elements of the single chain component are in the sequence [LC-L1-LC-L2-scBM]. Each heavy chain HC consists of VH, CH1, CH2 and CH3 immunoglobulin domains. The single chain component consists of a first light chain LC, which in turn consists of VL and CL immunoglobulin domains; a first linker L1; a second LC which is identical to the first; a second linker L2; and a single chain binding module in the form of an scFv.

FIG. 3 shows the result of SDS-PAGE analysis after purification of the indicated expressed molecules.

FIG. 4 is a diagram showing the binding of the indicated molecules to mTfR1, analyzed by indirect ELISA as described in Example 5.

FIG. 5 shows mTfR1 binding by the indicated molecules, measured using Octet biolayer interferometry as described in Example 6. A) mAb158-scLc-8D3-Ig.1; B) mAb158-scLc-8D3-Ig.2; C) mAb158-scLc-8D3-Ig.3; D) mAb158-scLc-8D3-Ig.4; E) mAb158-scLc-8D3-Ig.5; F) mAb158-scLc-Ig.6; G) 8D3 Fab; and H) mAb158 IgG.

FIG. 6 is a bar diagram showing the binding of indicated molecules to cEND cells, analyzed using flow cytometry as described in Example 7. MFI (mean fluorescence intensity) represents the amount of the tested molecule that was bound to cells.

FIG. 7 is a bar diagram showing the internalization of bispecific binding molecules by cells, presented as the percentage of positive cells (cEND) in the population captured using a fluorescently labelled antibody, as described in Example 7.

FIG. 8 is a diagram showing the binding of the indicated molecules to Aβ1-42, analyzed by indirect ELISA as described in Example 8.

FIG. 9 shows stability in plasma in vitro analyzed by Western blot as described in Example 9.

FIG. 10 is a series of bar diagrams showing concentrations at 24 h post i.v. dose of 10 nmol/kg of mAb158 IgG, mAb158-scLc-8D3-Ig.1, or mAb158-scLc-8D3-Ig.3 in C57BL/6J female mice in (A) plasma, (B) left brain hemisphere TBS-Triton extracts, both analyzed using MSD, and showing (C) the brain-to-plasma concentration ratios for the respective constructs. Circles represent individual data points and bars represent mean±SD. A one-way ANOVA with Tukey's post hoc test was performed. ****p<0.0001.

FIG. 11 is a series of confocal images from immunohistochemistry (IHC) analysis of cerebral cortex, using mAb158 control IgG (left panel), mAb158-scLc-8D3-Ig.1 (central panel) and mAb158-scLc-8D3-Ig.3 (right panel), 24 h after an i.v. dose of 10 nmol/kg in C57BL/6J WT female mice. mAb158-scLc-8D3-Ig.1 and mAb158-scLc-8D3-Ig.3 were detected in brain capillaries (arrows), parenchyma and around brain cells (arrowheads). Scale bar: 50 μm.

FIG. 12 shows mTfR1 binding by the indicated molecules measured using Octet biolayer interferometry as described in Example 6. A) mAb158-scLc-8D3-Ig.7; B) mAb158-scLc-8D3-Ig.8; C) mAb158-scLc-8D3-Ig.9; D) mAb158-scLc-8D3-Ig.10; E) mAb158-scLc-8D3-Ig.11; F) mAb158-scLc-Ig.12; G) mAb158-scLc-Ig.14; H) mAb158-scLc-Ig.15; and I) mAb158.

FIG. 13 is a series of diagrams showing the binding of the indicated molecules to Aβ1-42, analyzed by indirect ELISA as described in Example 8. A) mAb158-scLc-8D3-Ig.7, mAb158-scLc-8D3-Ig.8, mAb158-scLc-8D3-Ig.9, mAb158-scLc-8D3-Ig.11, mAb158-scLc-Ig.12 and mAb158-scLc-Ig.15; B) mAb158-scLc-8D3-Ig.10, mAb158-scLc-Ig.12, mAb158-scLc-Ig.14 and mAb158.

FIG. 14 shows binding to mTfR1 by the indicated molecules, measured using surface plasmon resonance as described in Example 12.

FIG. 15 shows mTfR1 binding by the indicated molecules, measured using Octet biolayer interferometry as described in Example 6. A) mAbB-scLc-8D3-Ig.2; B) mAbB-scLc-8D3-Ig.3; C) mAbB.

FIG. 16 shows mTfR1 binding by mAbB-scLc-8D3-Ig.1, measured using surface plasmon resonance as described in Example 13.

FIG. 17 shows Aβ binding by the indicated molecules, measured using surface plasmon resonance as described in Example 13. A) mAbB-scLc-8D3-Ig.1; B) mAbB-scLc-8D3-Ig.2; C) mAbB-scLc-8D3-Ig.3; D) mAbB.

FIG. 18 shows bar diagrams of binding molecule concentrations in plasma and brain 24 h after an i.v. dose of 40 nmol/kg of mAbB and mAbB-scLc-8D3-Ig.1 in 5×FAD mice and WT littermates in plasma (left) and in left brain hemisphere TBS-Triton extracts (middle), both analyzed using MSD as described in Example 14, as well as of the brain-to-plasma concentration ratios for the respective constructs and transgenes (right). Circles represent individual data points and bars represent mean±SD.

FIG. 19 shows wide-field fluorescence microscopy images of the brain distribution of i.v. dosed antibody detected by anti-hIgG1 secondary antibody in sagittal 5×FAD brain sections as described in Example 15. Lower panels show high magnification images from cerebral cortex (CTX), hippocampus (HC) and thalamus (TH) co-stained for Aβ plaques (detected by combined 6E10+4G8).

FIG. 20 shows the results of image analysis of the number of mAbB and mAbB-scLc-8D3-Ig.1 decorated amyloid beta plaques per field of view and colocalization (% of 6E10/4G8 plaques labeled with hIgG) in thalamus, measured as described in Example 15.

FIG. 21 shows hTfR1 binding by mAb158-scLc-15G11-1-Ig.1, measured using surface plasmon resonance as described in Example 16.

FIG. 22 is a diagram showing the binding of the indicated molecules to Aβ1-42 analyzed by indirect ELISA as described in Example 16.

### EXAMPLES

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment, but that the invention will include all embodiments falling within the scope of the appended claims.

The invention will be further illustrated by the following non-limiting Examples. They are offered for illustrative purposes only and are not intended to limit the invention in any manner. Those of skill in the art will readily recognize a variety of non-critical parameters which can be changed or modified to yield essentially the same results. Efforts have been made to ensure accuracy with respect to numbers used (e.g. amounts, temperatures, etc.), but some experimental error and deviations may be present. Unless otherwise indicated, the practice of the invention employs conventional methods of protein chemistry, biochemistry, recombinant DNA techniques and pharmacology, within the skill of the art. Such techniques are explained fully in the existing literature. Additionally, it will be apparent to one of skill in the art that the methods for protein engineering applied to certain linker designs herein can also be applied to other constructs described herein and contemplated by the present inventors to fall within the scope of the disclosure.

### Example 1

### Generation of Plasmids and Protein Expression

Proteins were expressed by transient transfection of Chinese hamster ovary cells (ExpiCHO™) or human embryonic kidney cells (Expi293F™) (Thermo Fisher Scientific).

For expression of protein, pcDNA3.4 plasmids (Thermo Fisher Scientific) were used and designed to comprise the following functional elements:
a promoter element from cytomegalovirus (CMV),
a murine kappa light chain signaling sequence (SS),
a gene to be expressed, and
a full stop using double stop codons, e.g. TGA, TAA.

### Example 2

### Overall Design and Amino Acid Sequences of Exemplary Bispecific Binding Molecules of the Disclosure

All binding molecules described in this Example were designed using the same antibody heavy chain (HC). More specifically, the heavy chain comprised a murine VH domain from the Aβ protofibril binding antibody mAb158, coupled to a murine CH1 domain of IgG2c subclass, in turn coupled to a human IgG1 CH2-CH3 part, all encoded on a pcDNA3.4 plasmid. In other words, the heavy chain was a chimeric construct comprising a murine VH-CH1 part (Fab) and a human CH2-CH3 part (Fc). The complete heavy chain amino acid sequence is given in SEQ ID NO:7, and is referred to herein as the “mAb158 heavy chain”.

Variation between the different constructs was achieved through the respective single chain component, in which different linker lengths were used to connect the elements of the continuous polypeptide chain making up the single chain component.

In each tested single chain component, the two identical antibody light chains LC were derived from the light chain of the Aβ protofibril binding antibody mAb158, and each LC consisted of a VL-CL part of the kappa class having the amino acid sequence SEQ ID NO:8.

Also in each tested single chain component, the single chain binding module scBM was a single chain variable fragment (scFv) derived from the transferrin receptor binding antibody 8D3 (Kissel et al (1998), Histochem Cell Biol 110:63-72) and having the amino acid sequence SEQ ID NO:9. This scFv, derived from 8D3 and used in the constructs, is sometimes denoted “8D3” herein for brevity.

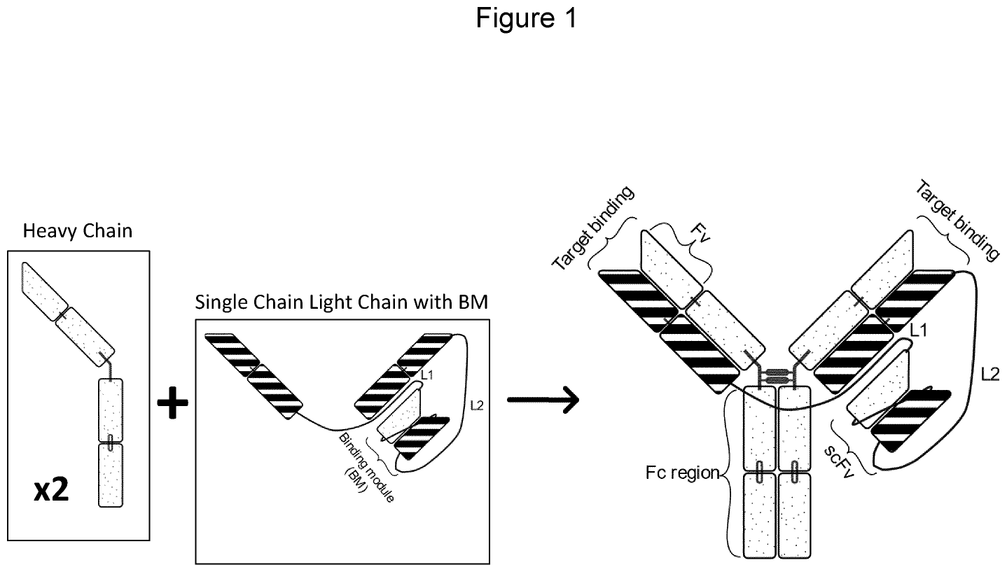

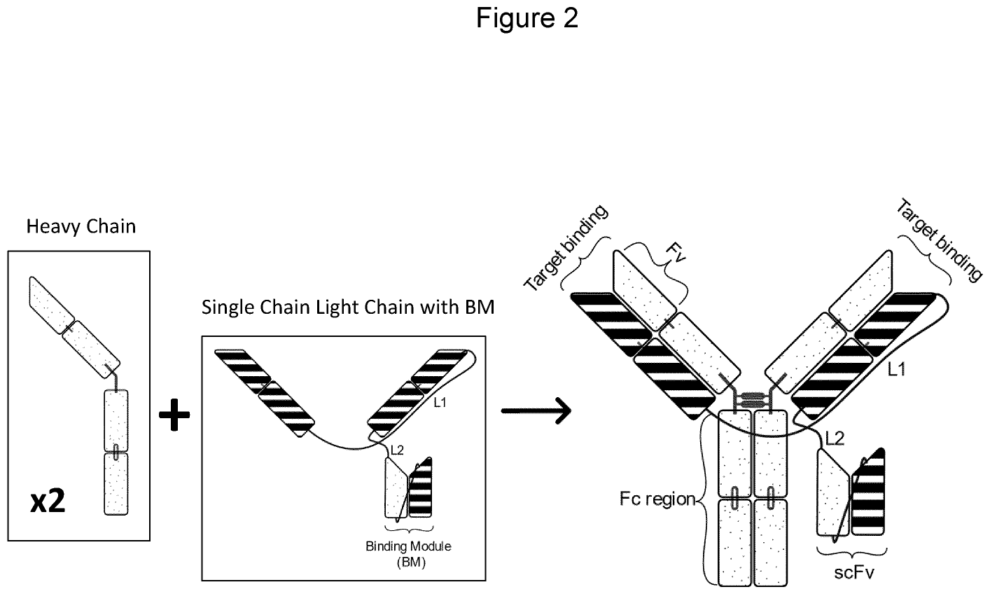

The different tested constructs are presented in Table 1, and were designed according to either the format depicted in FIG. 1 (mAb158-scLc-8D3-Ig.1, mAb158-scLc-8D3-Ig.2 and mAb158-scLc-8D3-Ig.3), the format depicted in FIG. 2 (mAb158-scLc-8D3-Ig.4 and mAb158-scLc-8D3-Ig.5) or as a negative control without any scBM element (mAb158-scLc-Ig.6).

TABLE 1

| | | | | Overall | |
|---|---|---|---|---|---|
| | | | Total number | single chain | Theoretical |
| | Linker L1 | Linker L2 | of linker | component | molecular |
| | (SEQ | (SEQ | amino acid | sequence | weight |
| Designation | ID NO) | ID NO) | residues | (SEQ ID NO) | (kDa) |
| mAb158-scLc-8D3-Ig.1 | 1 | 5 | 51 | 10 | 177 |
| mAb158-scLc-8D3-Ig.2 | 2 | 2 | 50 | 11 | 177 |
| mAb158-scLc-8D3-Ig.3 | 5 | 6 | 108 | 12 | 182 |
| mAb158-scLc-8D3-Ig.4 | 3 | 1 | 45 | 13 | 176 |
| mAb158-scLc-8D3-Ig.5 | 4 | 1 | 65 | 14 | 178 |
| mAb158-scLc-Ig.6 | 4 | — | 50 | 15 | 151 |

### Example 3

### Production and Purification of Designed Constructs

Bispecific binding molecules of the disclosure and controls were expressed by transient transfection of Chinese hamster ovary cells (ExpiCHO; Thermo Fisher Scientific) according to the manufacturer's instructions. An equimolar ratio of heavy and light chain plasmids was added to the cells during transfection. Cell culture supernatants containing expressed constructs were harvested 8-10 days after transfection by centrifugation at 3200×g for 10 min. The supernatants were stored frozen until purification.

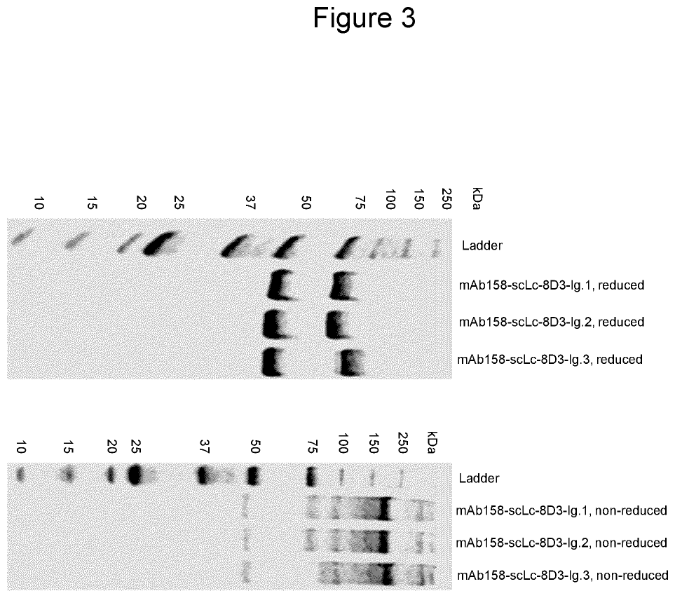

Upon purification, the frozen supernatants containing expressed constructs were thawed and filtered. Filtered supernatants were applied to a MabSelect SuRe column (Cytiva) which was subsequently washed with DPBS pH 7.4. Expressed binding molecules were eluted by application of 0.7% HAc pH 2.5, followed by immediate neutralization of the sample to pH 7.5. Purified samples were polished further by subjecting them to size exclusion chromatography (SEC; HiLoad 26/600 Superdex 200; Cytiva) in DPBS pH 7.4. The SEC purified, monomeric constructs were concentrated to 10 μM using centrifugal concentrators Amicon Ultra (30 MWCO, Millipore) and stored at −80° C. for further analysis. Each purified expressed construct was characterized using SDS-PAGE, size-exclusion chromatography (Superdex 200 Increase 3.2/300; Cytiva), endotoxin determination and UV protein determination. An example of a representative SDS-PAGE analysis of purified constructs is shown in FIG. 3.

### Example 4

### Antigen Production and Purification

A recombinant, murine transferrin receptor 1 (mTfR1) was produced by transient transfection of human embryonic kidney cells (Expi293F; Thermo Fisher Scientific) according to the manufacturer's instructions. The expression plasmid contained the extracellular domain of mTfR1 (amino acids 89-763; SEQ ID NO:16) fused to a His-tag for purification.

The mTfR1 protein was purified from the filtered cell culture supernatant. The supernatant was applied to a HisTrap Excel column (Cytiva), which was washed with 20 mM Tris, 200 mM NaCl and 5 mM imidazole. The protein was eluted with 20 mM Tris, 200 mM NaCl and 500 mM imidazole, followed by buffer exchange to DPBS pH 7.4 using a HiPrep 26/10 Desalting column (Cytiva). The protein was concentrated using a Amicon Ultra centrifugal concentrator (30 MWCO; Millipore). The protein was stored at −80° C. immediately upon purification to decrease aggregation. Analytical characterization of the protein was done by UV protein determination and SDS-PAGE, and it was concluded that the purification was successful.

### Example 5

### ELISA Analysis of Binding to Murine Transferrin Receptor

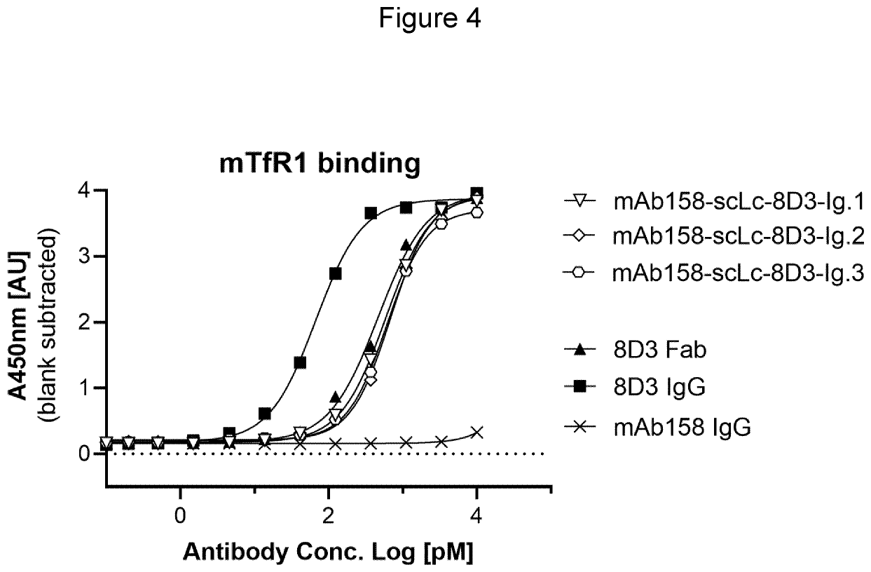

Binding of selected expressed constructs to mTfR1 was assessed by indirect ELISA (FIG. 4). In brief, half area 96-well plates (Corning, #3690) were coated with 1 μg/ml recombinant mTfR1, produced as described in Example 4, in PBS overnight at 4° C. The coated plates were blocked using Pierce protein-free blocking solution (Thermo Fisher Scientific, #37572) for 1 h at room temperature with shaking and washed four times in PBS containing 0.1% TWEEN-20. Serial dilutions (1:3) of expressed constructs in incubation buffer (1% BSA, 0.1% TWEEN-20 in PBS) were incubated for 1 h at room temperature. Following the four wash steps, bound test constructs were detected by addition of anti-mouse-IgG F(ab′)2-HRP antibody (Jackson Immuno Research, #115-035-006) at 1:1250 dilution in incubation buffer (1 h, room temperature). Following four wash steps, K-Blue® Aqueous TMB substrate (Neogen, #331177) was added to the wells for 15 min at room temperature before the reaction was stopped with 1:1 dilution of 0.5 M H₂SO₄. The optical density at 450 nm was recorded (Spark, Tecan) and background signal was subtracted before analysis. FIG. 4 shows mTfR1 binding for mAb158-scLc-8D3-Ig.1, mAb158-scLc-8D3-Ig.2 and mAb158-scLc-8D3-Ig.3 as measured by indirect ELISA. The affinity towards mTfR1 for all mAb158-scLc-8D3 variants are comparable to the monovalent 8D3 Fab control (with a light chain of SEQ ID NO:17 and a heavy chain of SEQ ID NO:18).

### Example 6

### Biolayer Interferometry Analysis of Binding to Murine Transferrin Receptor

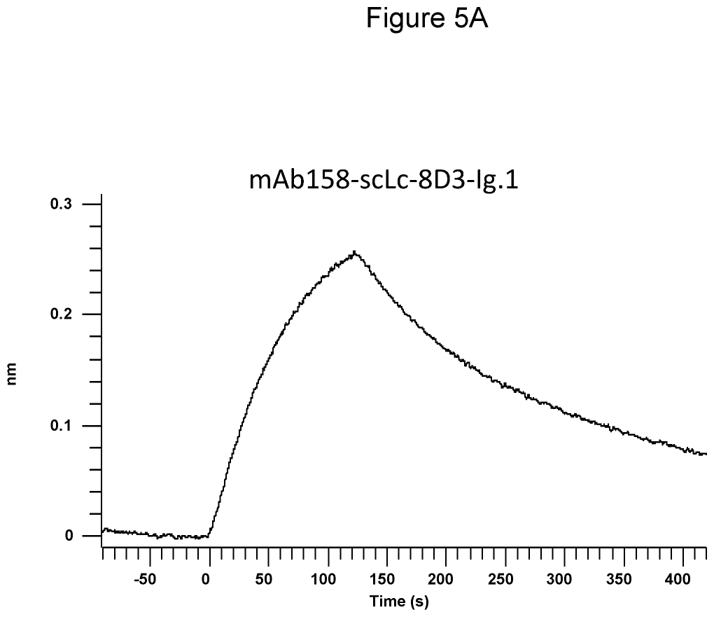

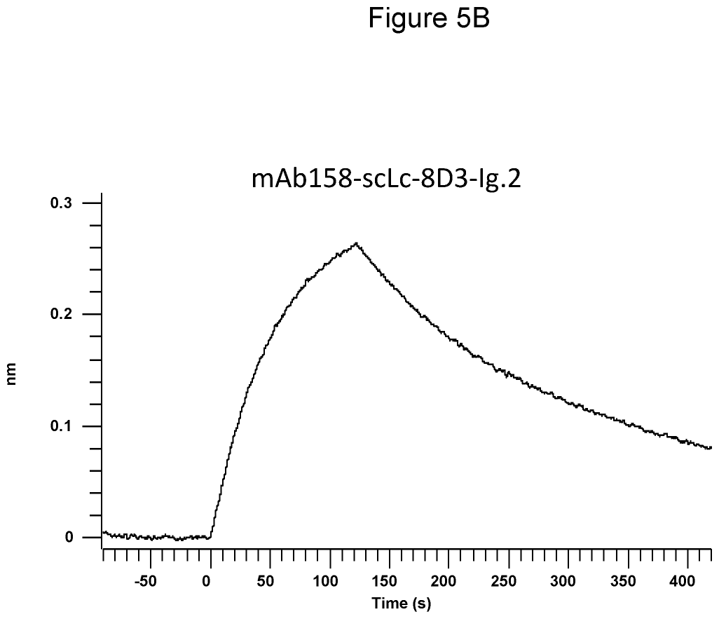

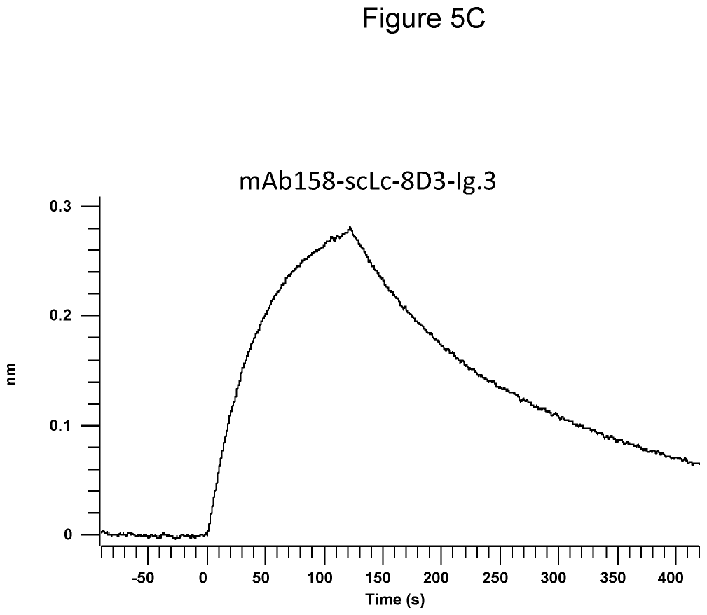

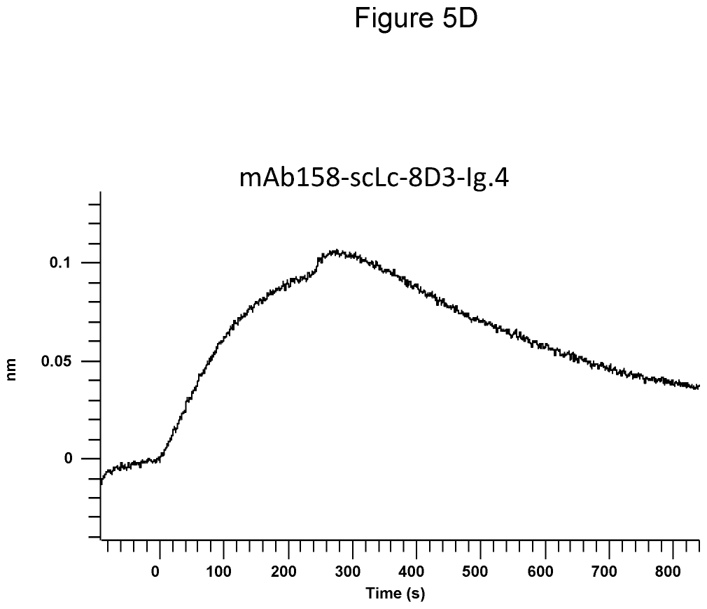

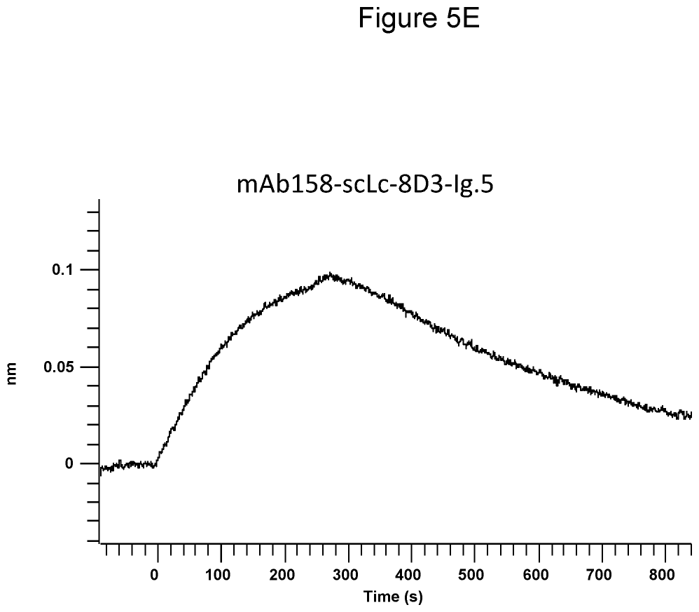

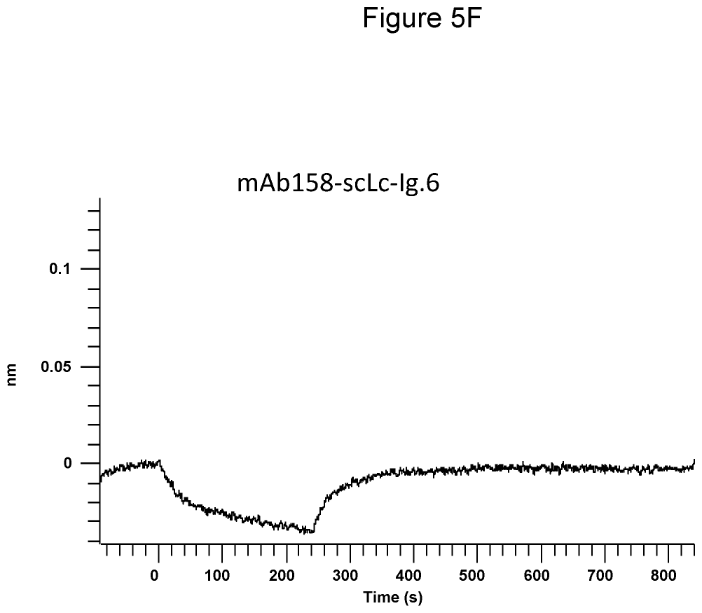

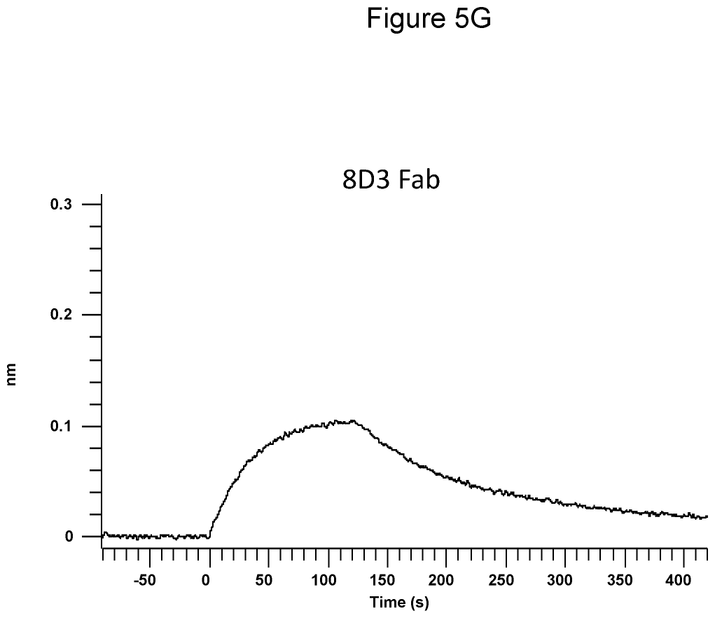

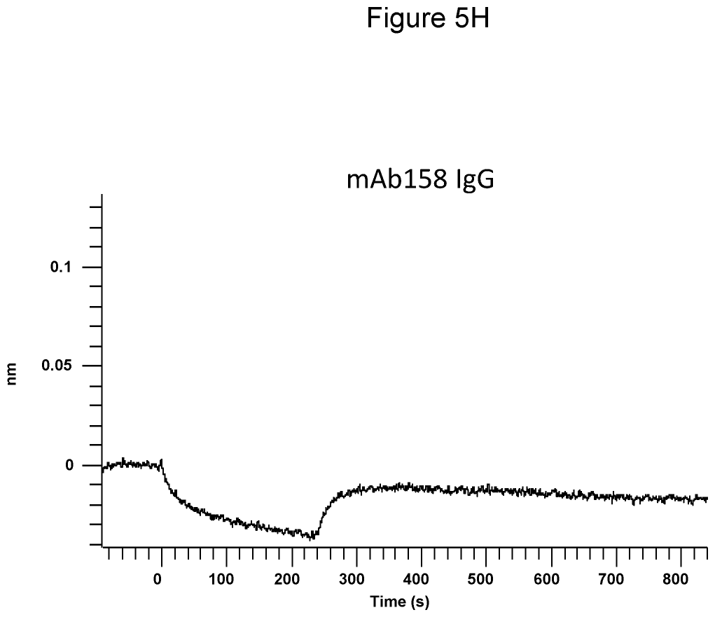

Binding of expressed constructs to mTfR1 was assessed by Bio-Layer Interferometry (Octet RED384, ForteBio). High-precision streptavidin sensors were loaded in two consecutive steps: first with 20 μg/ml of biotinylated human holo-transferrin (Sigma) during 180 s and then with 20 μg/ml recombinant mTfR1, produced as described in Example 4, during 180 s. Thereafter, association of the samples to the loaded sensors was measured for 120 s, followed by dissociation for 300 s. Binding was analyzed with samples diluted to 17.5 μg/ml for mAb158-scLc-8D3-Ig.1 and mAb158-scLc-8D3-Ig.2, to 18 μg/ml for mAb158-scLc-8D3-Ig.3 and to 4.9 μg/ml for 8D3 Fab (corresponding to 100 nM of respective binding molecule). The response from a buffer sample was subtracted. All samples were diluted in 1× Kinetics buffer (ForteBio). The same buffer was used for baseline and dissociation steps. The obtained binding curves are shown in FIG. 5, and show that mAb158-scLc-8D3-Ig.1, mAb158-scLc-8D3-Ig.2, mAb158-scLc-8D3-Ig.3, mAb158-scLc-8D3-Ig.4 and mAb158-scLc-8D3-Ig.5 all bind to the complex of holo-transferrin and mTfR1. mAb158-scLc-Ig.6, which lacks the TfR binding module, does not bind to the complex of holo-transferrin and mTfR1. As controls, the 8D3 Fab and the antibody mAb158 IgG were included.

### Example 7

### Binding to and Uptake by Cells

Binding and uptake mediated by TfR1 was measured on Immortalized Mouse Cerebral Capillary Endothelial Cells (cEND) (ABM, #T0290).

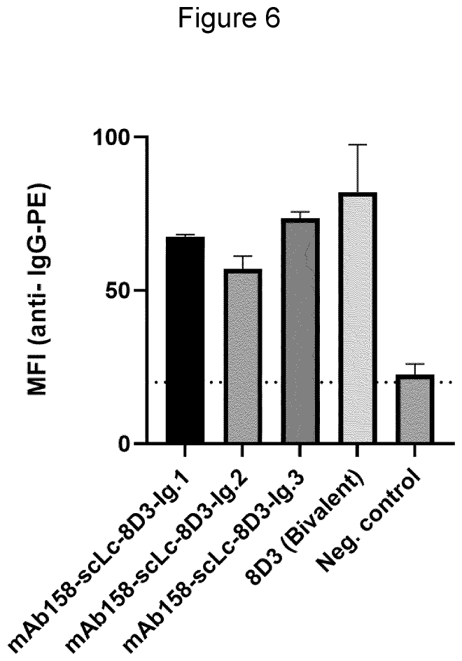

The pellet was resuspended in PBS and 200,000 cells per well were seeded in U-bottom 96-well Corning plates for flow cytometry staining. Cells were then washed with PBS and incubated with 100 nM of one of the mAb158-8D3 constructs mAb158-scLc-8D3-Ig.1, mAb158-scLc-8D3-Ig.2 and mAb158-scLc-8D3-Ig.3, control 8D3 IgG (light chain of SEQ ID NO:17, heavy chain of SEQ ID NO:19) or murine IgG control for 45 min at 4° C. After primary incubation, cells were washed twice with ice cold PBS, and mAb158-8D3 constructs were captured using a fluorescently labeled secondary anti-mouse IgG-PE antibody (BD Biosciences, 550589). After incubation, cells were washed twice with PBS and resuspended in 200 μl PBS. Cells were acquired in a BD FACSLyric flow cytometer system (BD Biosciences) and samples were analyzed using FCS Express software (De Novo Software). The measured mean fluorescence intensities are displayed in FIG. 6, which shows that the bispecific binding molecule constructs bind to mTfR1 expressed on cell surfaces.

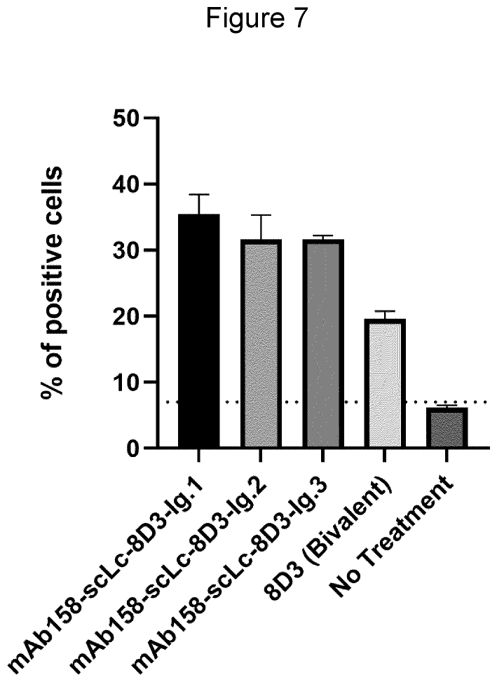

For measurement of construct internalization/uptake, cEND cells were cultured in T75 cell culture flasks for 2-3 days to >80% confluence. Cells were harvested using TrypIE reagents and washed with fresh cell medium by centrifugation at 1500 rpm for 5 min. Cells were resuspended in DPBS (Gibco) and seeded at a density of 300,000 cells/well in 96-well U-bottom plates. Cells were treated with 100 nM of mAb158-scLc-8D3-Ig.1, mAb158-scLc-8D3-Ig.2, mAb158-scLc-8D3-Ig.3 or positive control (bivalent 8D3 IgG) for 1 h at 37° C., 5% CO₂ or not treated. Then, cells were washed twice with ice cold PBS and permeabilized to access internalized constructs using BD Cytofix/Cytoperm reagent (BD Biosciences). Cells were stained with anti-mouse IgG-PE in 1% BD Perm/Wash for 45 min at RT. After staining, cells were washed twice with 1% BD Perm/Wash. Finally, cells were resuspended in PBS and acquired using BD FACSLyric flow cytometry. Samples were analyzed for PE positive population using FCS Express software. The percentage of positive cells, indicative of mTfR1-mediated uptake, are displayed in FIG. 7.

Taken together, these results demonstrate that the bispecific binding molecules mAb158-scLc-8D3-Ig.1, mAb158-scLc-8D3-Ig.2 and mAb158-scLc-8D3-Ig.3 bind to mTfR1 on cEND cells and are internalized to a greater degree than a bivalent 8D3 IgG.

### Example 8

### ELISA Measurement of Binding to Amyloid Beta (Aβ1-42)

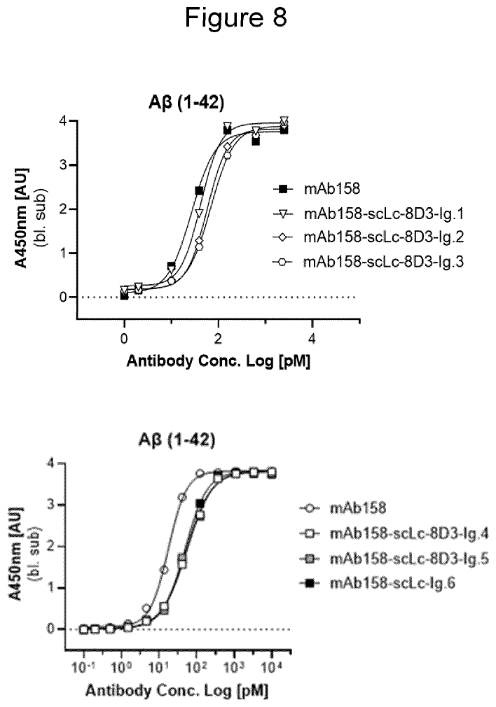

Binding of mAb158 IgG and mAb158-scLc-8D3 constructs to Aβ1-42 was assessed by indirect ELISA. Half area 96-well plates (Corning, #3690) were coated with 0.5 μg/ml recombinant Aβ1-42 (SEQ ID NO:20), and the analysis was conducted as described for mTfR1 in Example 5. The results are shown in FIG. 8, and demonstrate that the binding to Aβ1-42 by all tested constructs was comparable to binding by unmodified mAb158 IgG control, demonstrating that the binding to Aβ1-42 is maintained in the bispecific format, with the single chain component comprising a single chain binding module.

### Example 9

### Stability of Expressed Constructs in Plasma

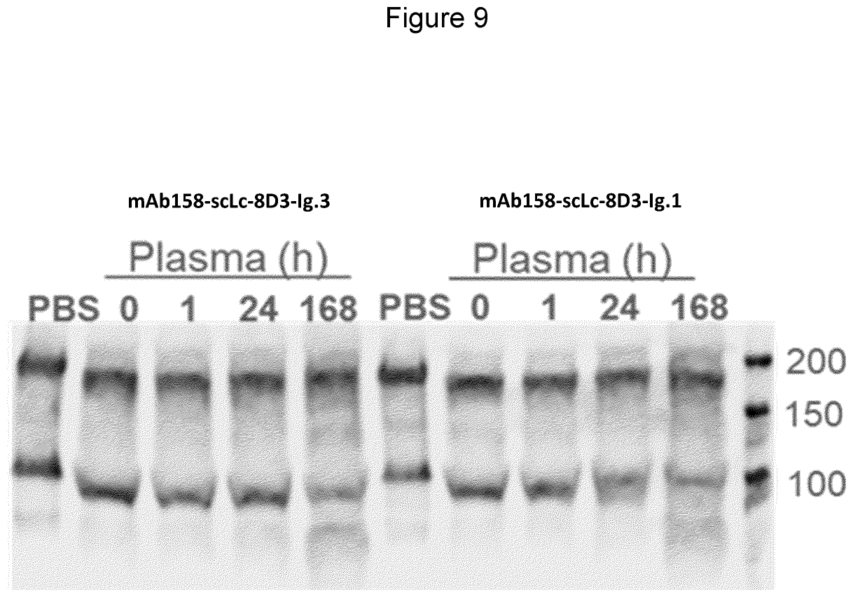

The stability in plasma of bispecific binding molecules mAb158-scLc-8D3-Ig.1 and mAb158-scLc-8D3-Ig.3 was determined by immunoblotting (western blot). Bispecific binding molecules were incubated in C57BL/6 mouse plasma or PBS for 0 h, 1 h, 24 h and 168 h at 37° C. (1.5 μg binding molecule diluted 1:10 in plasma or PBS). Samples were freeze-thawed twice and subjected to SDS-PAGE and western blot. In brief, 0.38 μg (2.5 μl) plasma/PBS samples were diluted in RIPA buffer and LDS Sample Buffer (Thermo Fisher Scientific, NP0007) and loaded onto a NuPAGE 4-12% Bis-Tris gel (Thermo Fisher Scientific, #NP0329). Following SDS-PAGE and transfer to nitrocellulose membranes (BioRad, #1704158), the molecules were detected by a goat anti-human IgG IRDye 800CW Secondary Antibody (LI-COR Biosciences, #926-32232). The results are shown in FIG. 9. The bispecific binding molecules were detected at the expected molecular weight ˜175 kDa, with a slight mobility shift between plasma and PBS. The western blot analysis shows that mAb158-scLc-8D3-Ig.1 and mAb158-scLc-8D3-Ig.3 are stable in mouse plasma in vitro for a period of at least 7 days.

### Example 10

### Brain Uptake of mAb158-scLc-8D3 Constructs In Vivo

To evaluate mTfR1-mediated brain uptake in vivo, the bispecific binding molecules mAb158-scLc-8D3-Ig.1 and mAb158-scLc-8D3-Ig.3, as well as mAb158 IgG (chimeric IgG1, human Fc) were injected intravenously (i.v.) into C57BL/6J female mice (n=3-5 mice per construct) at equimolar doses of 10 nmol/kg (corresponding to 1.82 mg/kg, 1.78 mg/kg and 1.50 mg/kg, respectively). Plasma and brain exposure were assessed 24 h after dose. The animals were anaesthetized using isoflurane and terminal blood samples were collected from the orbital plexus into BD Microtainer K2EDTA tubes. The samples were inverted and centrifuged at 2400×g for 10 min at 4° C. Plasma was extracted and transferred to Eppendorf tubes and frozen at −80° C. Immediately following blood sampling, the abdomen of the animals was cut open and a cannula (21 G) was inserted into the left ventricle of the heart. A small cut was made in the right atrium and transcardial perfusion was performed with a minimum of 50 ml of cold PBS. Following perfusion, brains were extracted, and the olfactory bulbs removed. The brains were separated into left and right hemispheres and cerebellum was removed from the left hemisphere, after which the left hemisphere was weighed and snap frozen on dry ice and stored at −80° C. until further preparation and analysis of the concentrations of injected constructs using a Meso Scale Discovery (MSD) based assay. The right hemispheres, with intact cerebellum, were placed in 4% formaldehyde and stored at 4° C. for 24 h, after which they were rinsed in cold PBS, transferred to cold 30% sucrose solution prepared in PBS and stored at 4° C. for further immunohistochemistry (IHC) processing.

For brain concentration measurements, frozen left hemispheres were thawed on ice and homogenized in TBS by automated bead homogenization. Triton was added to the homogenate to a final Triton concentration of 0.5% before centrifugation at 16 000×g, after which supernatants were collected.

Brain and plasma concentrations of mAb158-scLc-8D3-Ig.1, mAb158-scLc-8D3-Ig.3 and mAb158 IgG were determined using the MSD platform. A standard 96-well MSD plate (MSD, #L15XA-3) was coated with 0.5 μg/ml goat anti-human IgG, Fcγ fragment specific antibody (Jackson Immuno Research Europe Ltd, #109-005-098) diluted in 1×PBS (Fisher Scientific, #09-9400-100), in a volume of 50 μl/well. After incubation at 4° C. overnight, the plate was washed 4× in 1×PBS-TWEEN (Fisher Scientific, #09-9410-100) and blocked with BlockerA (MSD, #R93BA-4). Samples and corresponding standards, ranging from 83 pM to 0.02 pM in 1:4 dilution steps, were diluted in BlockerA and added in duplicates in a volume of 50 μl/well. After incubating for 2 h and 900 rpm at room temperature, the wells were washed four times. 50 μl SULFO-TAG conjugated anti-mouse antibody (MSD, R32AC-1) diluted to 0.5 μg/ml in BlockerA was added per well and the plate was incubated for another hour at room temperature and stirring at 900 rpm. After a 4× wash, 150 μl 2× MSD read buffer (MSD, #R92TC) was added to each well and the plate was read in an MSD SECTOR Imager. The concentration of the analytes in the samples were evaluated with the MSD workbench software, using a 4PL curve fitting algorithm and curve weighting 1/Y2 for the standard curve. Statistical analysis was performed in GraphPad Prism (v. 9.0.0) using one way ANOVA with Tukey's post hoc test.

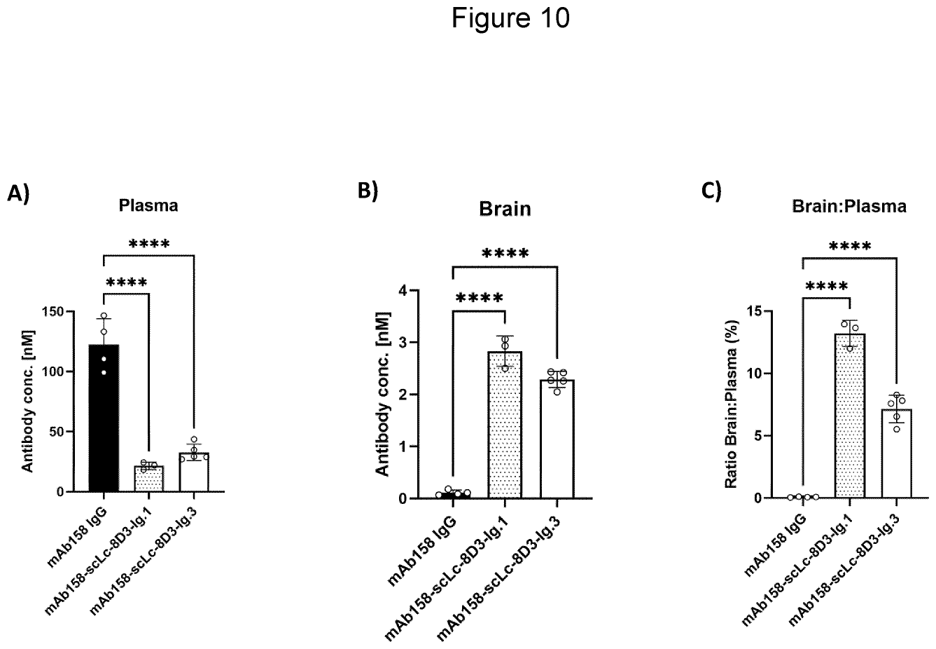

The results are shown in FIG. 10. As depicted in FIG. 10A, the plasma concentrations of both mAb158-scLc-8D3-Ig formats were well below the plasma concentrations of mAb158 IgG at 24 h after dose. The differences in the plasma data were expected, because the mAb158-scLc-8D3-Ig formats were suspected to be cleared faster from plasma compared to a regular IgG, due to the contribution of mTfR1-associated clearance. Despite the lower plasma concentrations, significantly higher brain concentrations (FIG. 10B, p<0.0001) were observed for both mAb158-scLc-8D3-Ig.1 and mAb158-scLc-8D3-Ig.3 in comparison to mAb158 IgG at 24 h after dose. The brain-to-plasma concentration ratios of mAb158-scLc-8D3-Ig.1 and mAb158-scLc-8D3-Ig.3 at 24 h also showed significantly enhanced brain exposure relative to plasma in comparison to mAb158 IgG (FIG. 10C, mAb158-scLc-8D3-Ig.1: p<0.0001; mAb158-scLc-8D3-Ig.3: p<0.0001). Taken together, the data supports mTfR1-mediated BBB transport, with resulting high brain concentrations of the tested bispecific binding molecules.

BBB transcytosis and brain uptake of mAb158-scLc-8D3-Ig.1 and mAb158-scLc-8D3-Ig.3 were further supported by qualitative IHC analysis. In brief, coronal brain sections, at a thickness of 20 μm, were obtained from PBS-perfused brain hemispheres using a cryostat (Microm HM 500 OM). The sections were collected on Superfrost plus slides (Menzel-Glsser, #J1800AMNZ) and air-dried prior to IHC. The brain sections were washed with PBS (pH 7.4) for 15 min and incubated in blocking buffer (5% BSA, 0.25% Triton-X in PBS) for 2 h at room temperature. To visualize i.v. dosed constructs, brain sections were incubated with a secondary goat anti-human IgG (heavy and light chain specific) conjugated to Alexa Fluor 488 (Invitrogen, #A11013) for 90 min at room temperature followed by 3×15 min wash in PBS. To visualize brain capillaries, a rabbit anti-collagen IV antibody (Bio-Rad, #2150-1470) was applied and detected by a secondary affinity-purified goat anti-rabbit IgG (heavy and light chain specific) conjugated to Alexa Fluor 568 (Invitrogen, #A-11011) (data not shown). Slides were mounted with Fluoromount-G (Invitrogen, #00-4958-02) for imaging analysis. Confocal images from cerebral cortex were captured using a Leica Stellaris 5 confocal system equipped with a HC PL APO 40×/1.25 GLYC motCORR CS2 objective (Leica, #11506423). Single confocal plane images at 2048×2048 pixel resolution were acquired at 600 Hz with a pinhole setting of 1 Airy unit.

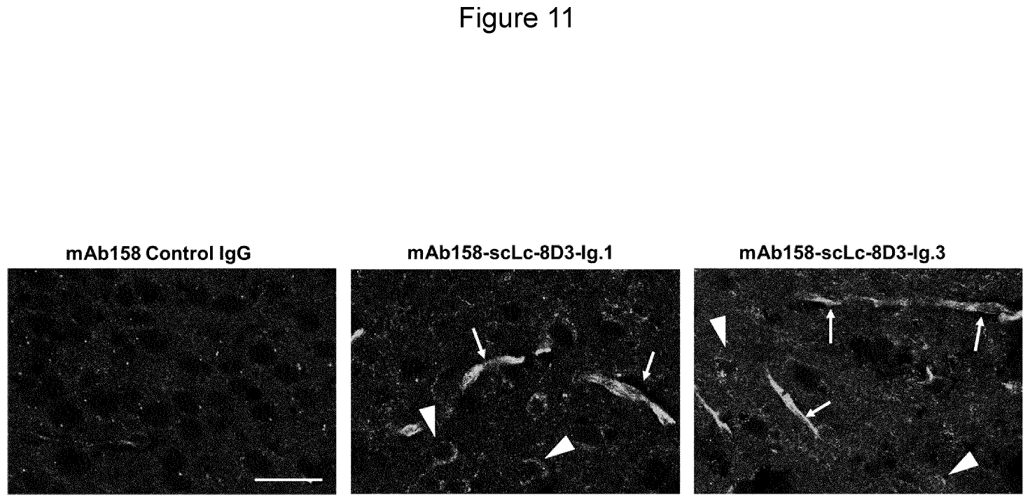

Minimal IHC signal was detected in brain sections from animals injected with mAb158 IgG (FIG. 11, left panel). In contrast, a distinct IHC signal was observed in brain capillaries along with an elevated parenchymal signal and immunodecorated brain cells in animals dosed with mAb158-scLc-8D3-Ig.1 and mAb158-scLc-8D3-Ig.3 (FIG. 11, central and right panels), indicative of mTfR1-mediated engagement and transcytosis over the BBB. Taken together, the MSD and IHC analysis demonstrate that the mAb158-scLc-8D3-Ig.1 and Ig.3 bispecific binding molecules confer enhanced brain exposure, relative to control mAb158 IgG.

### Example 11

### Design and Characterization of Additional mAb158-scLc-8D3 Constructs

All binding molecules described in this Example were created using the same antibody IgG1 heavy chain (HC) as in Example 2, i.e. SEQ ID NO:7.

Variation between the different constructs was achieved through the respective single chain component, in which different linker lengths were used to connect the elements of the continuous polypeptide chain making up the single chain component. All binding molecules described in this Example were designed using the same LC as in Example 2, i.e. SEQ ID NO:8.

The different constructs presented in this Example either contained the same single chain binding module scBM as in Example 2, i.e. SEQ ID NO:9 (present in mAb158-scLc-8D3-Ig.7, mAb158-scLc-8D3-Ig.8, mAb158-scLc-8D3-Ig.9, mAb158-scLc-8D3-Ig.10, mAb158-scLc-8D3-Ig.11 and mAb158-scLc-8D3-Ig.12), or a single chain binding module scBM also derived from 8D3 but with a reversed order of heavy and light chain, SEQ ID NO:21 (present in mAb158-scLc-8D3-Ig.13, mAb158-scLc-8D3-Ig.14 and mAb158-scLc-8D3-Ig.15).

The different tested constructs are presented in Table 2, and were designed according to the format depicted in FIG. 1, with the exception that mAb158-scLc-8D3-Ig.13, mAb158-scLc-8D3-Ig.14 and mAb158-scLc-8D3-Ig.15 had an scBM in which the order of the heavy and light chain variable regions had been reversed.

TABLE 2

| | | | | Overall | |
|---|---|---|---|---|---|
| | | | Total | single chain | Theoretical |
| | Linker L1 | Linker L2 | number of | component | molecular |
| | (SEQ | (SEQ | linker | sequence | weight |
| Designation | ID NO) | ID NO) | AA | (SEQ ID NO) | (kDa) |
| mAb158-scLc-8D3-Ig.7 | 22 | 22 | 20 | 24 | 175 |
| mAb158-scLc-8D3-Ig.8 | 1 | 1 | 30 | 25 | 176 |
| mAb158-scLc-8D3-Ig.9 | 23 | 23 | 40 | 26 | 176 |
| mAb158-scLc-8D3-Ig.10 | 2 | 22 | 35 | 27 | 176 |
| mAb158-scLc-8D3-Ig.11 | 2 | 1 | 40 | 28 | 176 |
| mAb158-scLc-8D3-Ig.12 | 2 | 23 | 45 | 29 | 177 |
| mAb158-scLc-8D3-Ig.13 | 2 | 22 | 35 | 30 | 176 |
| mAb158-scLc-8D3-Ig.14 | 2 | 1 | 40 | 31 | 176 |
| mAb158-scLc-8D3-Ig.15 | 2 | 23 | 45 | 32 | 177 |

The bispecific binding molecules were expressed and purified as described in Example 3.

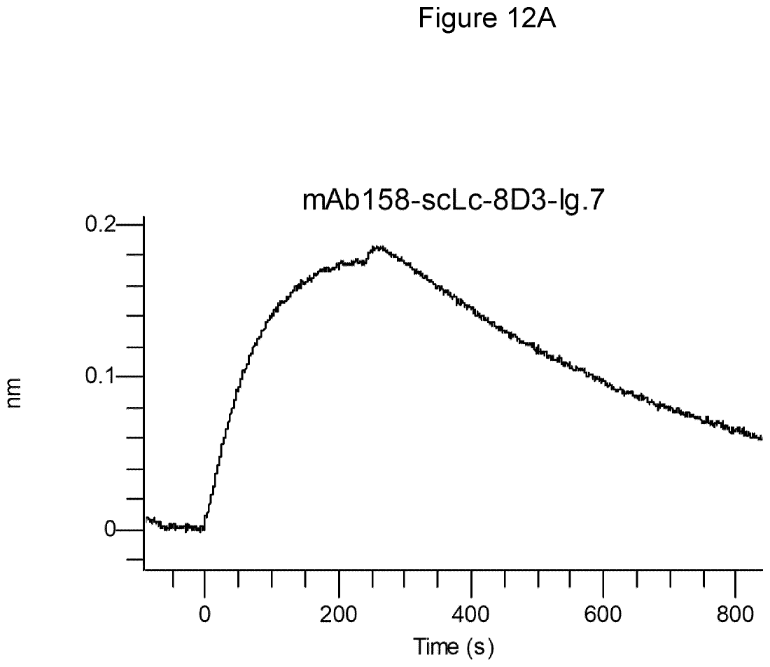

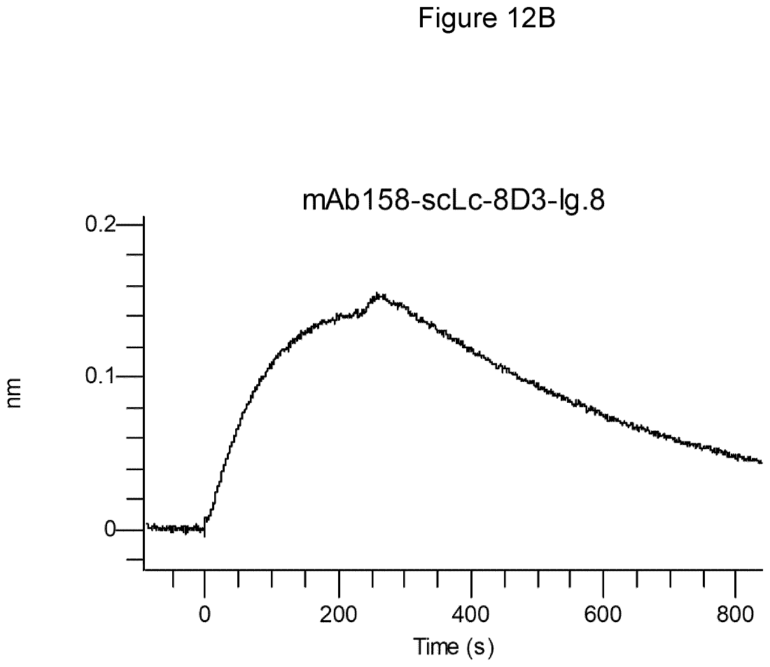

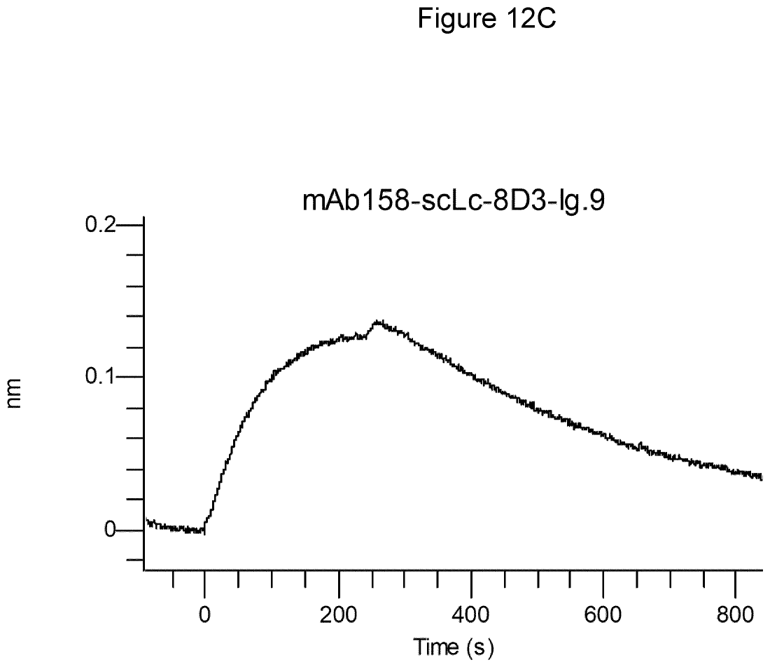

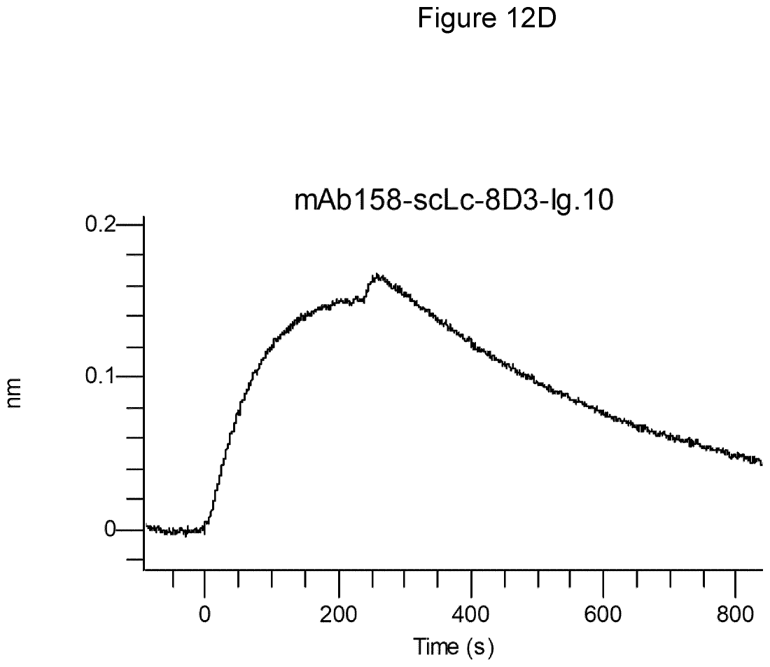

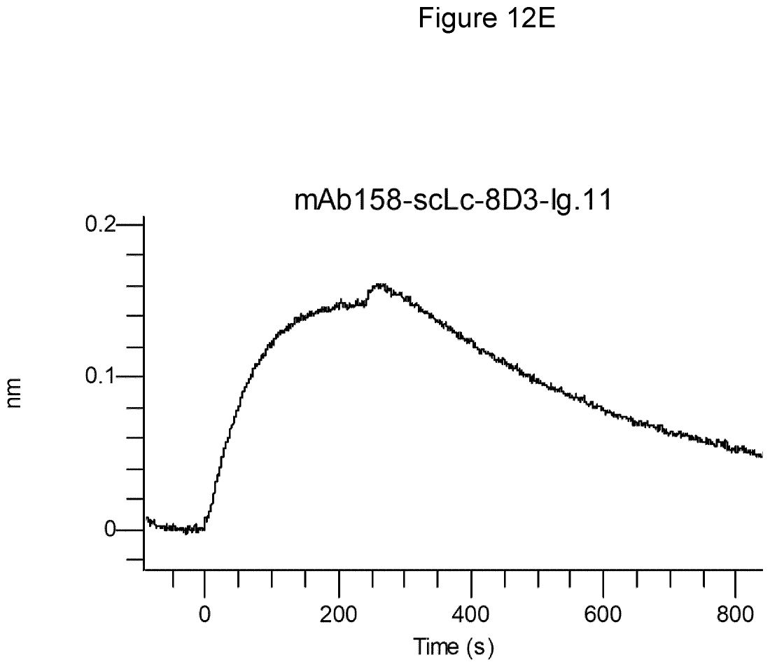

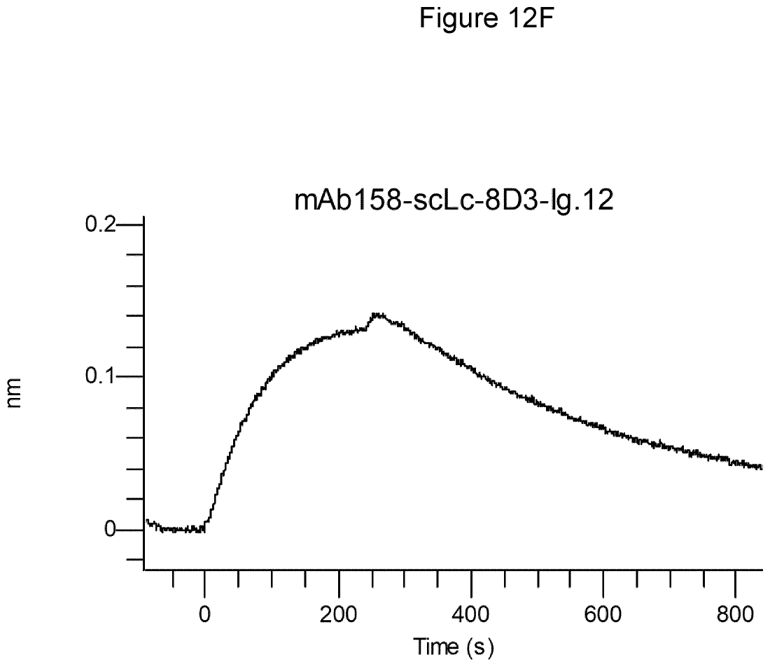

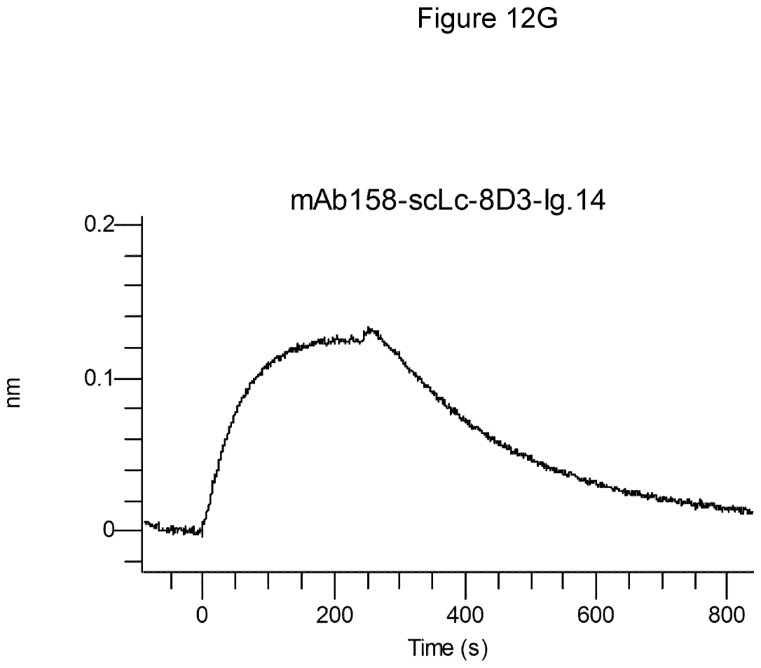

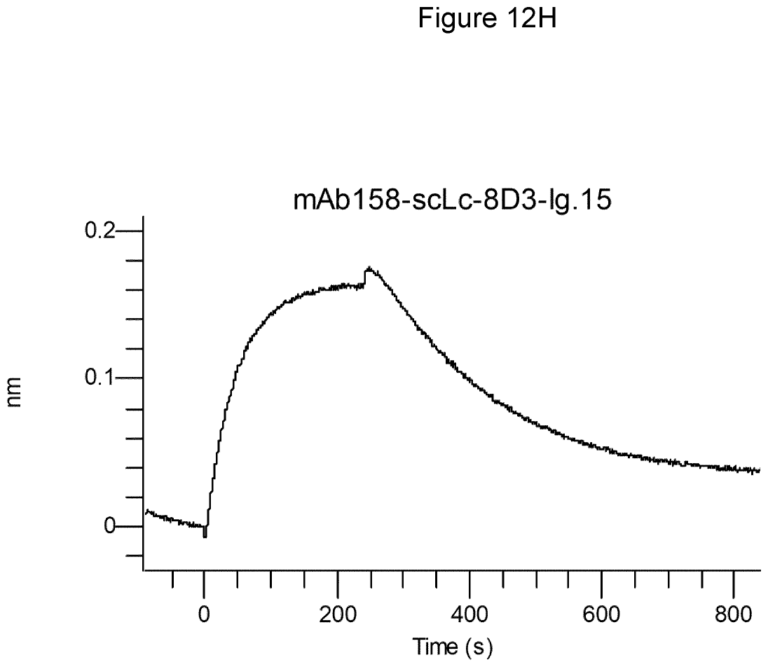

Binding of the expressed constructs to mTfR1 was assessed similarly as described in Example 6, except that the concentrations during loading were 10 μg/ml, the duration of loading steps were 200 s and 300 s, and the concentration of analytes was 50 nM. The obtained binding curves are shown in FIG. 12. All the tested constructs bind to mTfR1 (FIGS. 12A-12H). The antibody mAb158 without scBM was included as a negative control (FIG. 12I).

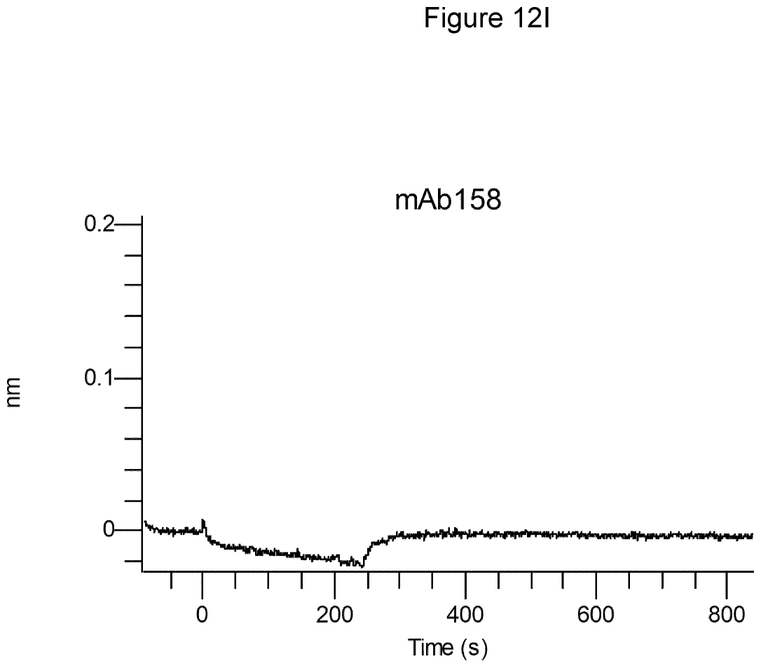

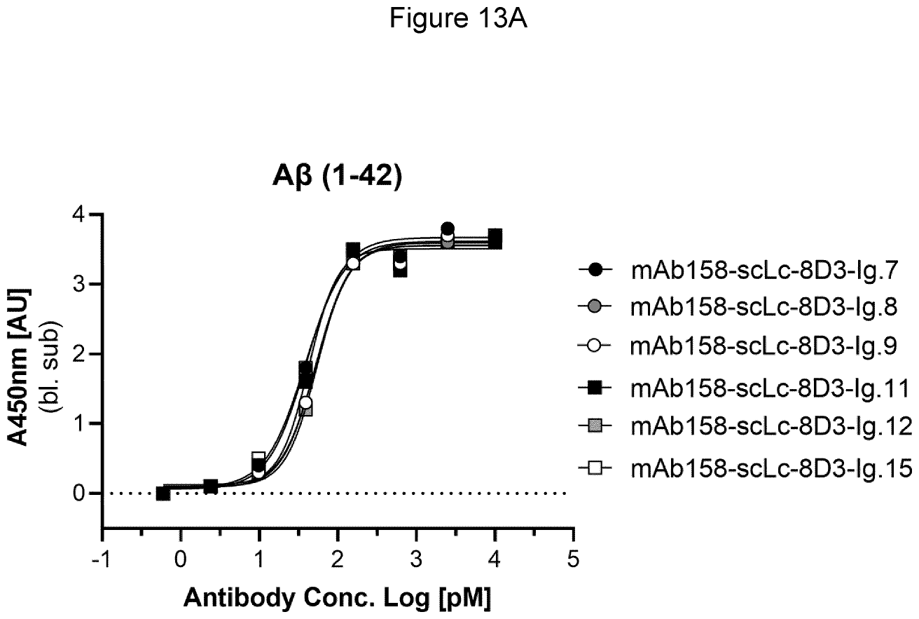

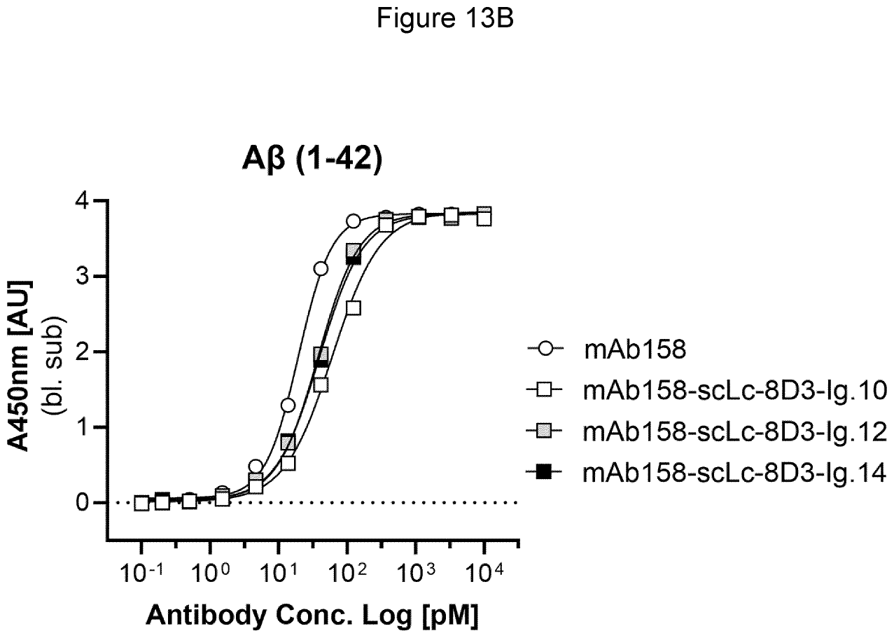

Binding of a selection of expressed constructs to Aβ1-42 was assessed by indirect ELISA as described in Example 8. The results are shown in FIG. 13, and demonstrate that binding to Aβ1-42 by all the tested constructs was comparable to binding by unmodified mAb158 control lacking the 8D3 scBM. The experiment demonstrates that binding to Aβ1-42 is maintained in the bispecific format by the Fab arms of the standard antibody configuration.

### Example 12

### Investigation of Monovalent Binding for Selected Constructs to TfR1

Monovalent binding interaction for mAb158-scLc-8D3-Ig.8 against murine TfR1 was measured using surface plasmon resonance (Biacore 8K, Cytiva). A Cm5 sensor chip (Cytiva, #BR100399) was immobilized with 3 μg/ml of mTfR1 using the amine coupling kit type 2 (Cytiva, #BR100633) according to the manufacturer's instruction. A 2-fold dilution series in HBS-EP+(Cytiva, #BR100669) starting at 50 nM of analyte was injected in duplicates over the immobilized ligand and the interaction was measured using the single cycle kinetics method with a contact time of 160 s at a flow rate of 70 μl/ml followed by a dissociation time of 1000 s.

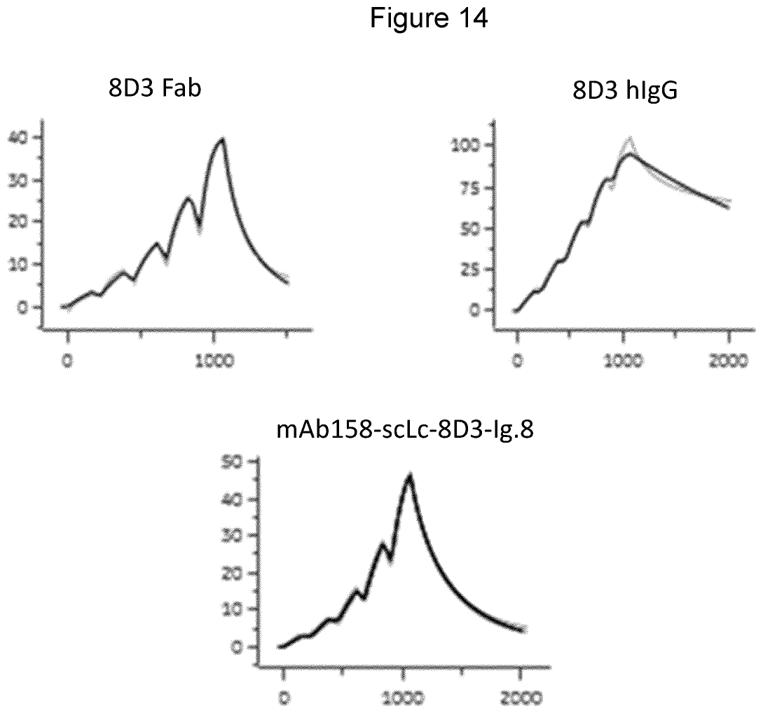

The resulting sensorgrams are shown in FIG. 14, and confirm that mAb158-scLc-8D3-Ig.8, i.e. a binding molecule according to the disclosure, binds monovalently to mTfR1. As controls, a monovalent 8D3 Fab was compared to a standard bivalent 8D3 hIgG. The diagrams clearly indicate a bivalent binding profile for 8D3 hIgG compared to the monovalent 8D3 Fab.

### Example 13

### Design and Characterization of Further Bispecific Binding Molecules According to the Disclosure

The binding molecules described in this Example were designed starting from a different Aβ binding antibody from the mAb158 used in the previous Examples.

The heavy chain (HC) of the bispecific binding molecules of this experiment comprised a murine VH domain from an Aβ binding antibody denoted mAbB herein, coupled to a human CH1-CH3 part (mAbB-scLc-8D3-Ig.1 and mAbB-scLc-8D3-Ig.2) or to a murine CH1 and a human CH2-CH3 (mAbB-scLc-8D3-Ig.3). In other words, the heavy chain was a chimeric construct comprising a murine VH part and a human CH1-CH3 part, or comprising a murine VH-CH1 part and a human CH2-CH3 part.

In each tested single chain component, the two identical antibody light chains LC were derived from the murine VL domain of the light chain of the Aβ binding antibody mAbB, coupled to a human CL part of the kappa class (mAbB-scLc-8D3-Ig.1 and mAbB-scLc-8D3-Ig.2) or to a murine CL part (mAbB-scLc-8D3-Ig.3).

All the different constructs presented in this Example contained the same single chain binding module scBM as in Example 2, i.e. SEQ ID NO:9.

The different tested constructs are presented in Table 3, and were designed according to the format depicted in FIG. 1.

TABLE 3

| | | | Total | Theoretical |
|---|---|---|---|---|
| | Linker L1 | Linker L2 | number of | molecular |
| | (SEQ | (SEQ | linker | weight |
| Designation | ID NO) | ID NO) | AA | (kDa) |
| mAbB-scLc-8D3-Ig.1 | 1 | 1 | 30 | 174 |
| mAbB-scLc-8D3-Ig.2 | 5 | 6 | 108 | 182 |
| mAbB-scLc-8D3-Ig.3 | 5 | 6 | 108 | 182 |

The bispecific binding molecules mAbB-scLc-8D3-Ig.1, mAbB-scLc-8D3-Ig.2 and mAbB-scLc-8D3-Ig.3 were expressed and purified as described in Example 3.

Binding of the expressed constructs to mTfR1 was assessed essentially as described in Example 6 for biolayer interferometry (Octet) and in Example 12 for surface plasmon resonance (Biacore).

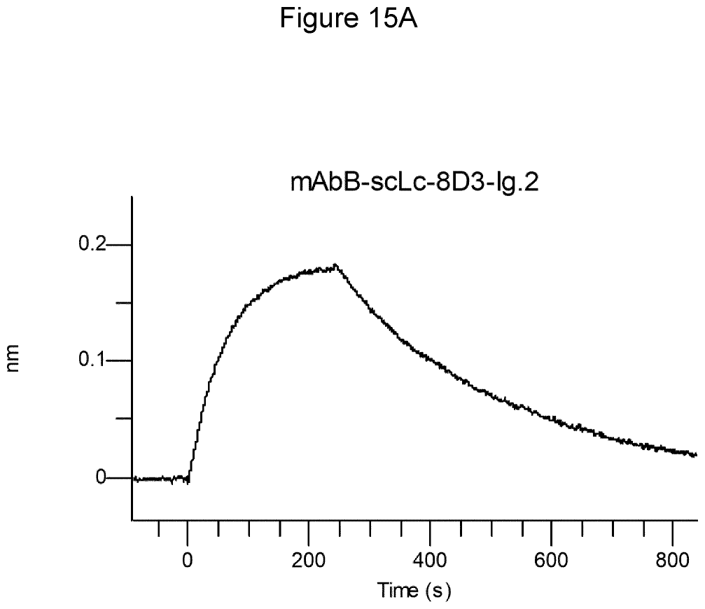

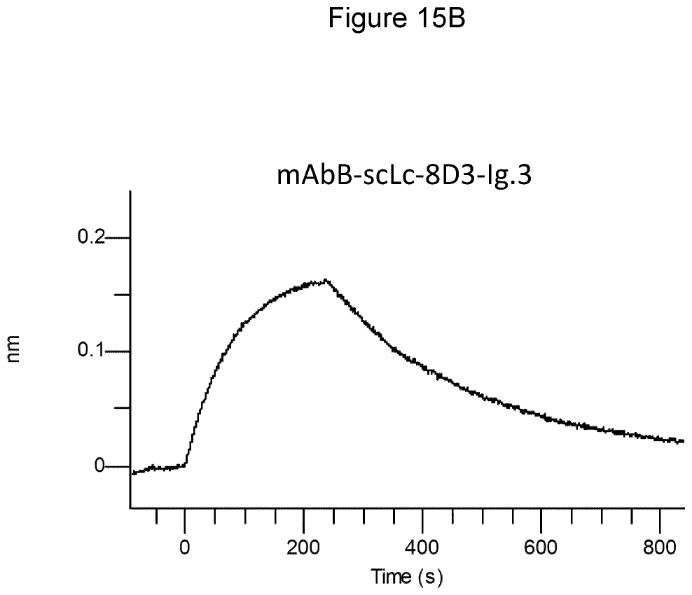

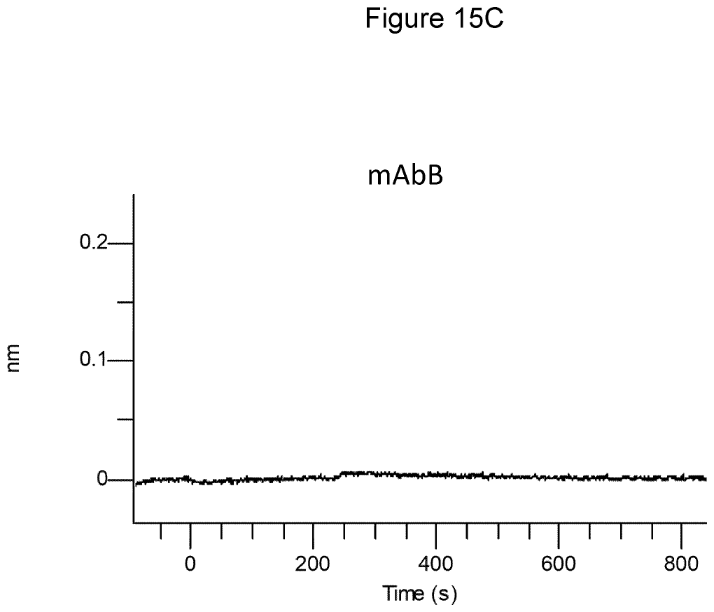

Representative results from the Octet measurements of binding to mTfR1 are shown in FIG. 15 for the bispecific binding molecules mAbB-scLc-8D3-Ig.2 and mAbB-scLc-8D3-Ig.3, and for the negative control mAbB, i.e. mAbB as a standard antibody format without any TfR1 binding module.

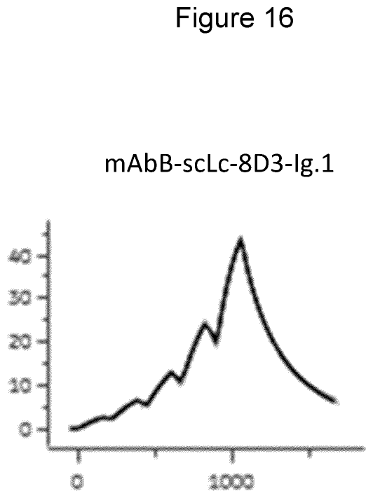

Representative results from the Biacore SCK measurements of binding to mTfR1 are shown in FIG. 16 for the bispecific binding molecule mAbB-scLc-8D3-Ig.1.

Binding of the expressed constructs to Aβ was evaluated using a Biacore 8K instrument (Cytiva) according to standard procedures. Single cycle kinetics (SCK) with binding molecules immobilized on a CM5 chip were used to measure the binding to the target. For measurements, 5 μg/ml of analyte binding molecule was immobilized on the chip. The Aβ target was then injected over the chip using a 2-fold dilution in five steps starting at 250 nM. Regeneration of the surface between cycles was done by injecting 30 μl 10 mM glycine-HCl, pH 1.7. The binding data was fitted to a 1:1 interaction model. 1×HBS-EP+(Cytiva, cat. no. BR100669) was used to dilute binding molecules and target antigens. Experiments were performed at 25° C.

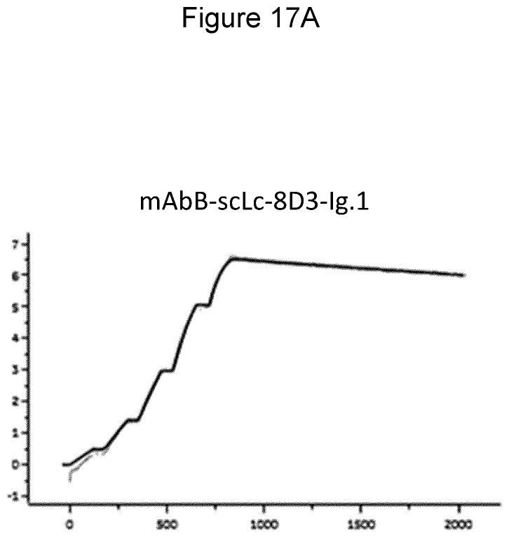

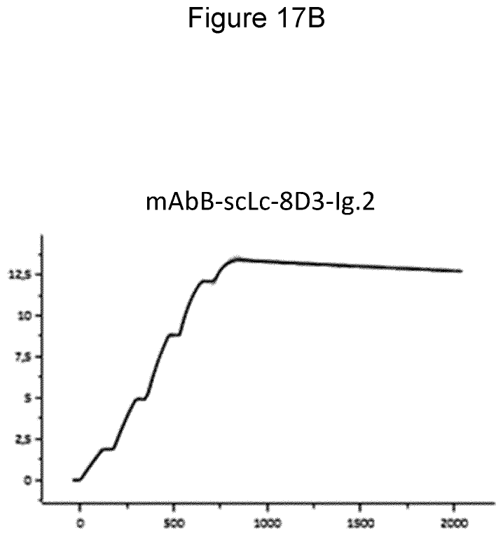

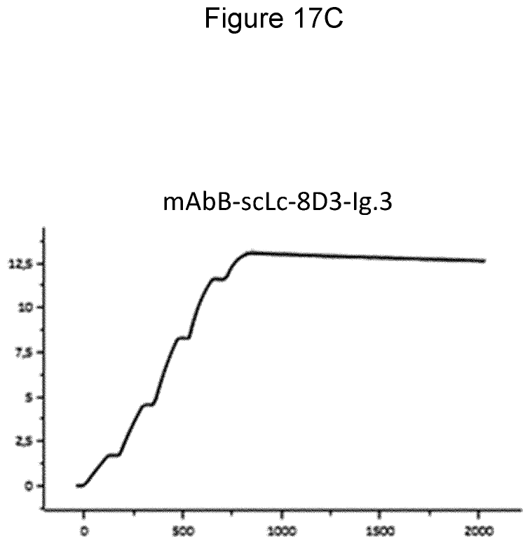

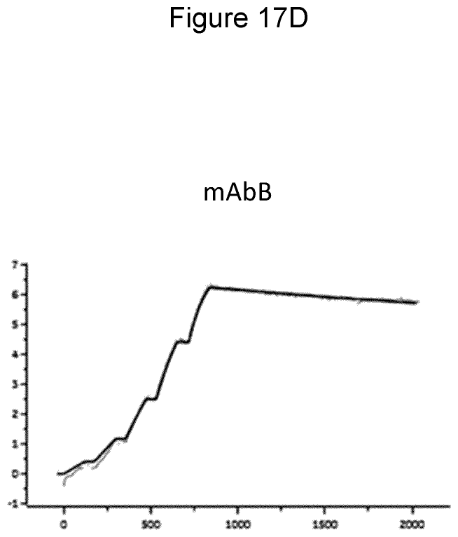

Representative results from the Biacore SCK measurements of binding to Aβ are shown in FIG. 17. Binding of mAbB-scLc-8D3-Ig.1, mAbB-scLc-8D3-Ig.2 and mAbB-scLc-8D3-Ig.3 to their Aβ target was assessed by Biacore and was found to be comparable to the binding of mAbB in the standard IgG format.

### Example 14

### Target Binding In Vivo by a Bispecific Binding Molecule of the Disclosure

To further evaluate target engagement in vivo, mAbB and mAbB-scLc-8D3-Ig.1 produced as described in Example 13 were investigated in B6SJL-Tg 5×FAD mice (Northwestern University). The 5×FAD mouse model is an Alzheimer's disease (AD) model with mice expressing human APP and PSEN1 transgenes with a total of five AD-linked mutations, including the Swedish (K670N/M671L), Florida (I716V), and London (V717I) mutations in APP, and the M146L and L286V mutations in PSEN1. 5×FAD mice and wild type (WT) littermates, at an age of 7-8 months, were intravenously (i.v.) injected with mAbB and mAbB-scLc-8D3-Ig.1 at equimolar doses of 40 nmol/kg (corresponding to 5.8 mg/kg and 7.0 mg/kg, respectively). Plasma and brain exposure to the respective binding molecule were assessed at 24 h post dose in both 5×FAD and WT mice, as well as binding to brain parenchymal Aβ plaques in 5×FAD mice. Terminal blood samples were collected by heart puncture into MiniCollect® K2EDTA tubes from all animals. The samples were inverted and centrifuged at 2400×g for 10 min at 4° C. Plasma was extracted and transferred to LoBind Eppendorf tubes and frozen at −80° C. Immediately following blood sampling, the animals were perfused with 0.9% saline and 4% paraformaldehyde (PFA; pH 7.4) or with 0.9% saline only, for brain immunohistochemistry (IHC) or antibody exposure analysis, respectively. Following perfusion, brains were extracted and the olfactory bulbs removed. The brains were separated into left and right hemispheres and cerebellum was removed from the left hemisphere in the animals perfused with saline only, after which the left hemisphere was weighed and snap frozen on dry ice and stored at −80° C. until further concentration measurements were performed using Meso Scale Discovery (MSD). The right hemispheres, and left for the animals perfused with both saline and PFA, with intact cerebellum, were post-fixed by immersion in freshly prepared 4% PFA (pH 7.4) for 2 h at room temperature, after which they were rinsed in cold PBS and transferred to 15% sucrose/PBS and stored at 4° C. until sunk to the bottom of the tubes. The hemispheres were transferred to cryomolds, embedded in OCT medium, frozen in dry ice-cooled isopentane and stored at −80° C.

For brain concentration measurements, frozen left hemispheres were thawed on ice and homogenized in TBS by automated bead homogenization. Triton was added to the homogenate to a final Triton concentration of 0.5% before centrifugation at 16 000× g, after which supernatants were collected.

Brain and plasma concentrations of mAbB and mAbB-scLc-8D3-Ig.1 were determined using the MSD platform. A sandwich set-up was used in standard 96-well MSD plates (MSD, #L15XA-3) coated with goat anti-human IgG (0.5 μg/ml, Fcγ specific antibody, Jackson Immuno Research Europe Ltd, #109-005-098) diluted in 1×PBS (Fisher Scientific, #09-9400-100). Plates were blocked for 1 h in BlockerA (MSD, #R93BA-4) before being incubated with samples and standards for 2 h. A 1 h incubation step of a mouse anti-human IgG1 (0.5 μg/ml, Mabtech, #3850-1-1000) was included, followed by 1 h incubation of SULFO-TAG conjugated anti-mouse antibody (0.5 μg/ml, MSD, #R32AC-1). MSD read buffer (MSD, #R92TC) was added to the wells before reading the plates in an MSD SECTOR Imager. Between each incubation step, a 4× wash in 1×PBS-Tween (Fisher Scientific, #09-9410-100) was performed. All binding molecules except the coating antibody were diluted in BlockerA.

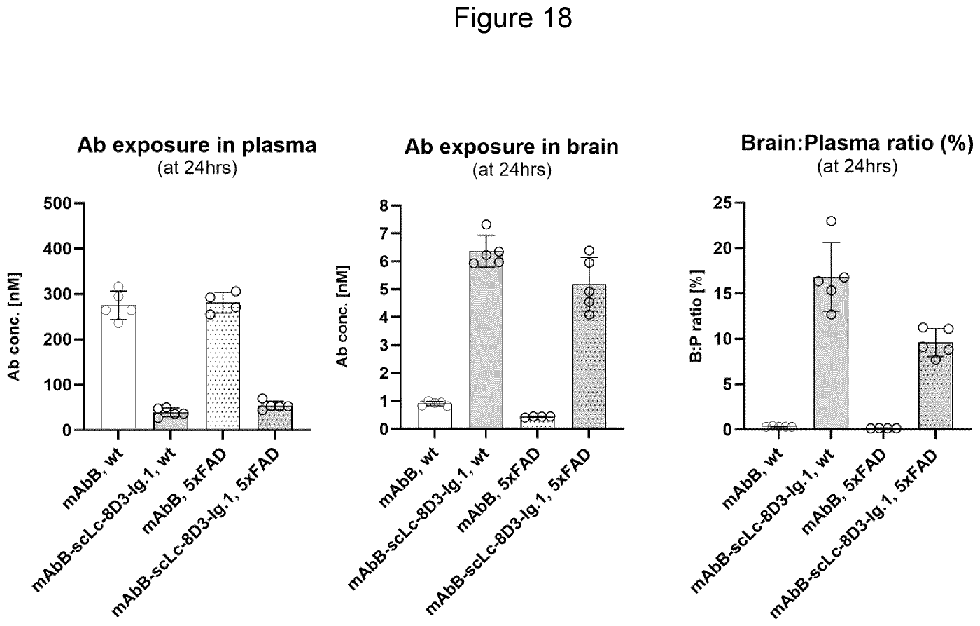

The results are shown in FIG. 18. The bispecific binding molecule of mAbB with the 8D3 scBM, mAbB-scLc-8D3-Ig.1, displays a lower plasma exposure but higher brain exposure in comparison to mAbB in a traditional antibody format. In addition, mAbB-scLc-8D3-Ig.1 provides a higher brain to plasma ratio (B:P ratio).

### Example 15

### Immunohistochemical Analysis of In Vivo Binding by a Bispecific Binding Molecule of the Disclosure

Transcytosis through the blood brain barrier and binding to the Aβ target by the disclosed binding molecule mAbB-scLc-8D3-Ig.1 was further evaluated by IHC in the transgenic mouse model 5×FAD.

In brief, 10 μm thick sagittal brain sections were obtained from 5×FAD mice using a cryostat (Leica CM1950 or CryoStar NX70). Collection of sections started at a level approximately 0.50 mm laterally from the midline and extended through the hemisphere. Sections were stored at −20° C.

Unspecific binding was blocked with M.O.M. blocking reagent (MKB-2213, Vector Laboratories) in 0.1% TritonX-100/PBS for 60 min, and rinsed in PBS. Amyloid plaques were labeled with the primary antibodies 6E10 (Biozym Scientific, B803001, 1:1000) and 4G8 (Biozym Scientific, B800701), 1:500, in M.O.M. diluent at 4° C. overnight. Intravenously dosed constructs comprising human IgG chains were detected by anti-human IgG (H+L) Alexa Fluor 647 (Jackson Immuno Reseach, 709-605-149), and the two primary mIgG antibodies were visualized by donkey anti-mouse IgG (H+L), DyLight 550 (Thermo Scientific, SA5-10167). Cell nuclei were labelled with DAPI. Slides were mounted in Mowiol and sections were imaged in a Zeiss automatic microscope AxioScan Z1 scanner with high aperture lenses, equipped with a Zeiss Axiocam 506 mono and a Hitachi 3CCD HV-F202SCL camera and Zeiss ZEN 3.3 software.

Automated IHC analysis of plaque numbers and co-localization in thalamus (N=5 mice/treatment) was conducted in Fuji ImageJ (v1.53c; Schindelin et al (2012), Nat Meth 9(7):676-682) using Colocalization Threshold & Analyze Particle plugins. Statistical analysis was performed using a one-way ANOVA with Tukey's post hoc test.

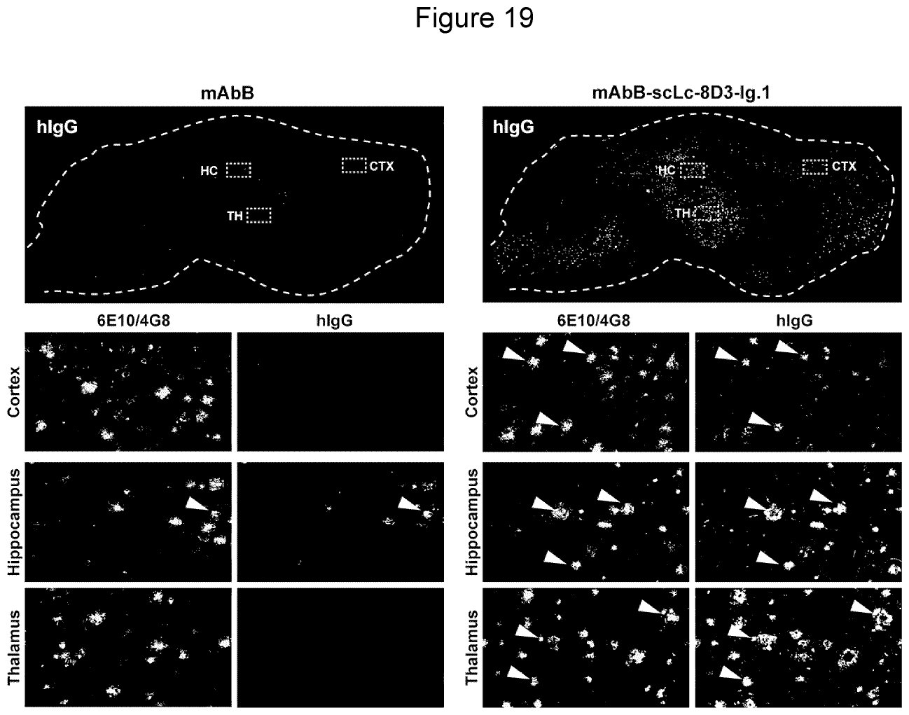

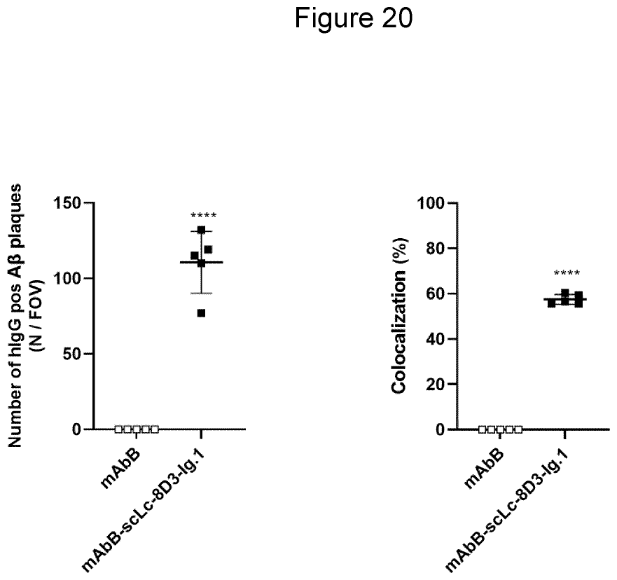

The results are shown in FIGS. 19 and 20. The bispecific binding molecule of mAbB with the 8D3 scBM, mAbB-scLc-8D3-Ig.1, shows a much stronger target engagement compared to mAbB in a traditional antibody format. The arrows indicate where mAbB-scLc-8D3-Ig.1 and mAbB respectively have reached the target (based on 6E10/4G8 staining) in the brain based on the hIgG staining. The hIgG staining is much more extensive and stronger for mAbB-scLc-8D3-Ig.1 compared to that seen for mAbB.

### Example 16

### Design and Characterization of a Further Bispecific Binding Molecule of the Disclosure

The binding molecule described in this Example was designed similarly to the previously studied binding molecules, but using a different single chain binding module scBM.

The heavy chain (HC) of the bispecific binding molecules of this experiment comprised a murine VH domain from mAb158, coupled to a human CH1-CH3 part. The complete heavy chain amino acid sequence is given in SEQ ID NO:33.

The two identical antibody light chains LC were derived from the VL domain of the light chain of mAb158, coupled to a human CL part of the kappa class. The amino acid sequence of each LC is represented by SEQ ID NO:34.

The single chain binding module scBM used here was a single chain variable fragment (scFv) derived from the transferrin receptor binding antibody 15G11-1 (Yu et al (2014) Sci Transl Med 6(261):261ra154) and having the amino acid sequence SEQ ID NO:35.

The tested construct was designed according to the format depicted in FIG. 1, and is presented in Table 4 including its linker sequences.

TABLE 4

| | | | | Overall | |
|---|---|---|---|---|---|
| | | | Total | single chain | Theoretical |
| | Linker L1 | Linker L2 | number of | component | molecular |
| | (SEQ | (SEQ | linker | sequence | weight |
| Designation | ID NO) | ID NO) | AA | (SEQ ID NO) | (kDa) |
| mAb158-scLc-15G11-1-Ig.1 | 1 | 1 | 30 | 36 | 175 |

The bispecific binding molecule mAb158-scLc-15G11-1-Ig.1 was expressed and purified as described in Example 3.

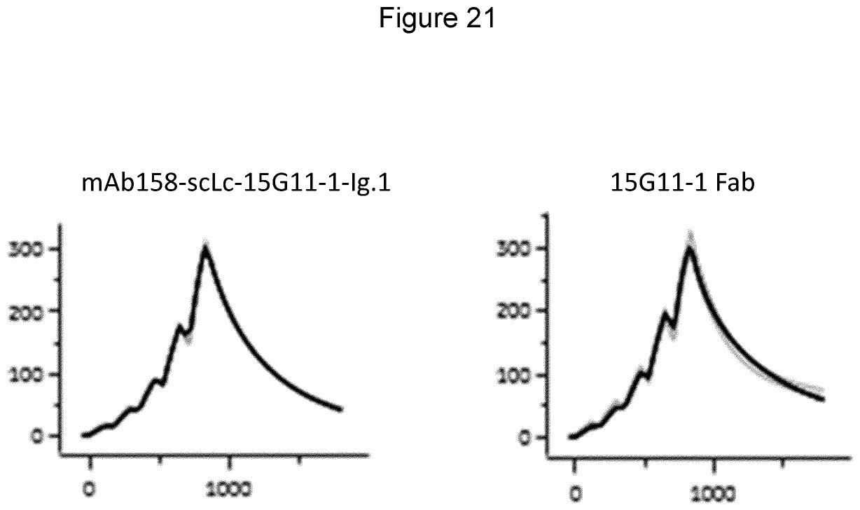

Binding to hTfR1 was assessed as described in Example 12 except that hTfR1 was used for immobilization and 3 M MgCl₂ was used for regeneration. The results are shown in FIG. 21, and demonstrate that the affinity towards hTfR1 for the expressed variants was comparable to that of the 15G11-1 Fab control.

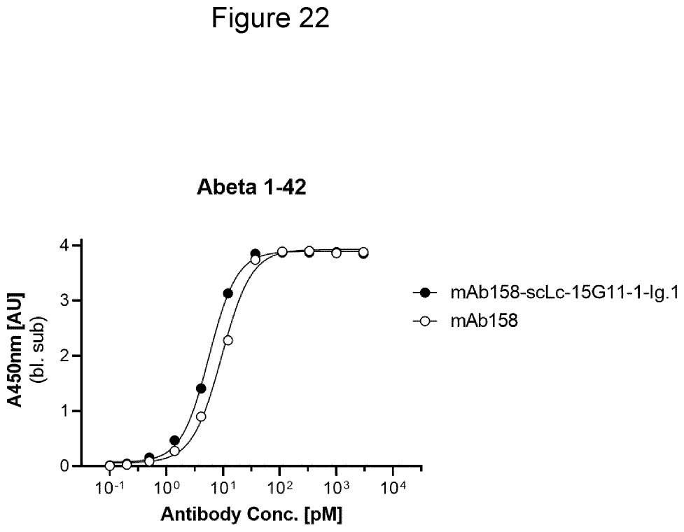

Binding of the expressed constructs to Aβ1-42 was assessed by indirect ELISA as described in Example 8. The results are shown in FIG. 22, and demonstrate that binding to Aβ1-42 by mAb158-scLc-15G11-1-Ig.1 was comparable to binding by the unmodified mAb158 control.

### ITEMIZED LISTING OF EMBODIMENTS

1. Bispecific binding molecule, comprising the following three polypeptide chains:
(A) two identical antibody heavy chains (HC) derived from a monoclonal antibody with affinity for a first target present in the brain of a mammal; and
(B) a single chain component, which comprises the following five elements in a continuous polypeptide chain:
   i) two identical antibody light chains (LC) derived from said monoclonal antibody with affinity for said first target;
   ii) one single chain binding module (scBM) with affinity for a second target which mediates transport of the bispecific binding molecule through the blood-brain barrier; and
   iii) two amino acid linkers L1 and L2, wherein said light chains (LC) and said single chain binding module (scBM) are separated by said linkers L1 and L2, thus forming a sequence from the N terminus to the C terminus selected from the group consisting of
      [LC-L1-scBM-L2-LC],
      [LC-L1-LC-L2-scBM] and
      [scBM-L1-LC-L2-LC].

2. Bispecific binding molecule according to item 1, wherein the sequence of elements from the N terminus to the C terminus in said single chain component is selected from the group consisting of
[LC-L1-scBM-L2-LC],
[LC-L1-LC-L2-scBM]

3. Bispecific binding molecule according to item 2, wherein the sequence of elements from the N terminus to the C terminus in said single chain component is [LC-L1-scBM-L2-LC].

4. Bispecific binding molecule according to item 2, wherein the sequence of elements from the N terminus to the C terminus in said single chain component is [LC-L1-LC-L2-scBM].

5. Bispecific binding molecule according to any preceding item, wherein said first target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), beta-secretase 1 (BACE1), superoxide dismutase (SOD), huntingtin, transthyretin, P-secretase 1, epidermal growth factor, epidermal growth factor receptor 2, Tau, phosphorylated Tau or fragments thereof, apolipoprotein E4, CD20, prion protein, leucine rich repeat kinase 2, parkin, presenilin 2, gamma secretase, death receptor 6, amyloid-β precursor protein, p75 neurotrophin receptor, neuregulin and caspase 6.

6. Bispecific binding molecule according to item 5, wherein said first target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), Tau, phosphorylated Tau or fragments thereof and apolipoprotein E4.

7. Bispecific binding molecule according to item 6, wherein said first target is selected from the group consisting of amyloid-β peptide or derivatives or fragments thereof, alpha-synuclein or derivatives or fragments thereof and TAR DNA-binding protein 43 (TDP-43) or derivatives or fragments thereof.

8. Bispecific binding molecule according to any preceding item, wherein said monoclonal antibody with affinity for said first target is an anti-A3 antibody selected from the group consisting of lecanemab, gantenerumab, aducanumab, donanemab, PBD-C06 and KHK6640.

9. Bispecific binding molecule according to any one of items 1-7, wherein said monoclonal antibody with affinity for said first target is the anti-alpha-synuclein antibody ABBV0805.

10. Bispecific binding molecule according to any preceding item, wherein said scBM is of a type selected from the group consisting of scFv, scFab, VHH and VNAR.

11. Bispecific binding molecule according to item 10, wherein said scBM is selected from the group consisting of scFv and scFab.

12. Bispecific binding molecule according to item 11, wherein said scBM is a scFv.

13. Bispecific binding molecule according to item 11, wherein said scBM is a scFab.

14. Bispecific binding molecule according to any preceding item, wherein said second target is selected from the group consisting of transferrin receptor 1 (TfR1), insulin receptor (InsR), insulin-like growth factor 1 receptor (IGF-1R), low density lipoprotein receptor-related protein 8 (Lrp8), low density lipoprotein receptor-related protein 1 (Lrp1), CD98, transmembrane protein 50A (TMEM50A), glucose transporter 1 (Glut1), basigin (BSG) and heparin-binding epidermal growth factor-like growth factor.

15. Bispecific binding molecule according to item 14, wherein said second target is selected from the group consisting of transferrin receptor 1 (TfR1), insulin receptor (InsR), insulin-like growth factor 1 receptor (IGF-1R) and low density lipoprotein receptor-related protein 8 (Lrp8).

16. Bispecific binding molecule according to item 15, wherein said second target is transferrin receptor 1.

17. Bispecific binding molecule according to any preceding item, wherein at least one of said amino acid linkers L1 and L2 is a flexible linker.

18. Bispecific binding molecule according to item 17, wherein both of said amino acid linkers L1 and L2 are flexible linkers.

19. Bispecific binding molecule according to any one of items 17-18, wherein said flexible linker(s) comprise(s) glycine, serine, alanine and/or threonine residues.

20. Bispecific binding molecule according to item 19, wherein said linker(s) has a general formula selected from (GₙSₘ)ₚ and (SₙGₘ)ₚ, wherein, independently, n=1-7, m=0-7, n+m≤8 and p=1-10.

21. Bispecific binding molecule according to any preceding item, wherein at least one of said amino acid linkers L1 and L2 is between 10 and 50 amino acid residues long, such as between 10 and 30 amino acid residues long, such as between 15 and 25 amino acid residues long or between 10 and 20 amino acids long.

22. Bispecific binding molecule according to item 21, wherein both of said amino acid linkers L1 and L2 are between 10 and 50 amino acid residues long, such as between 10 and 30 amino acid residues long, such as between 15 and 25 amino acid residues long or between 10 and 20 amino acids long.

23. Bispecific binding molecule according to any preceding item, wherein said amino acid linkers L1 and L2 are of the same length.

24. Bispecific binding molecule according to any one of items 1-22, wherein said amino acid linkers L1 and L2 are of different length.

25. Bispecific binding molecule according to item 24, wherein said amino acid linker L1 is longer than said amino acid linker L2.

26. Bispecific binding molecule according to item 24, wherein said amino acid linker L2 is longer than said amino acid linker L1.

27. Pharmaceutical composition, comprising a bispecific binding molecule according to any preceding item and a pharmaceutically acceptable carrier or excipient.

28. A bispecific binding molecule according to any one of items 1-26 or a composition according to item 27 for use in treatment, such as for use in therapeutic treatment or for use in prophylactic treatment.

29. A bispecific binding molecule according to any one of items 1-26 or a composition according to item 27 for use in diagnosis in vivo or prognosis in vivo.

30. A bispecific binding molecule or composition for use according to any one of items 28-29, wherein said therapy, prophylaxis, in vivo diagnosis or in vivo prognosis is with respect to a neurodegenerative disorder, for example a disorder selected from Alzheimer's disease and other disorders associated with Aβ protein aggregation, traumatic brain injury (TBI), Lewy body dementia (LBD), Down's syndrome (DS), amyotrophic lateral sclerosis (ALS), frontotemporal dementia, tauopathy, systemic amyloidosis, atherosclerosis, Parkinson's disease (PD), Parkinson's disease dementia (PDD), the Lewy body variant of Alzheimer's disease, multiple system atrophy, psychosis, schizophrenia, Creutzfeldt-Jakob disease, Huntington's disease, and familial amyloid neuropathy.

31. A bispecific binding molecule or composition for use according to item 30, wherein said therapy, prophylaxis, in vivo diagnosis or in vivo prognosis is with respect to a disorder selected from Alzheimer's disease and other disorders associated with Aβ protein aggregation, Lewy body dementia (LBD), Down's syndrome (DS), amyotrophic lateral sclerosis (ALS), frontotemporal dementia, tauopathy, Parkinson's disease (PD), Parkinson's disease dementia (PDD) and the Lewy body variant of Alzheimer's disease.

32. A bispecific binding molecule or composition for use according to item 31, wherein said therapy, prophylaxis, in vivo diagnosis or in vivo prognosis is with respect to a disorder selected from Alzheimer's disease and other disorders associated with Aβ protein aggregation, Lewy body dementia (LBD), amyotrophic lateral sclerosis (ALS) and Parkinson's disease (PD).

33. A bispecific binding molecule or composition for use according to item 32, wherein said therapy, prophylaxis, in vivo diagnosis or in vivo prognosis is with respect to Alzheimer's disease.

34. A bispecific binding molecule or composition for use according to any one of items 28-29, wherein said therapy, prophylaxis, in vivo diagnosis or in vivo prognosis is with respect to a disorder selected from brain cancer, multiple sclerosis and lysosomal storage diseases.

35. A method of therapeutic or prophylactic treatment of a mammal having, or being at risk of developing, a disorder, said method comprising administering to said mammal a therapeutically effective amount of a bispecific binding molecule according to any one of items 1-26 or a composition according to item 27.

36. A method according to item 35, wherein said disorder is a neurodegenerative disorder, for example a neurodegenerative disorder as defined in any one of items 30-33.

37. A method according to item 35, wherein said disorder is as defined in item 34.

1. Bispecific binding molecule, comprising the following three polypeptide chains:
(A) two identical antibody heavy chains (HC) derived from a monoclonal antibody with affinity for a first target present in the brain of a mammal; and
(B) a single chain component, which comprises the following five elements in a continuous polypeptide chain:
i) two identical antibody light chains (LC) derived from said monoclonal antibody with affinity for said first target; ii) one single chain binding module (scBM) with affinity for a second target which mediates transport of the bispecific binding molecule through the blood-brain barrier; and
iii) two amino acid linkers L1 and L2, wherein said light chains (LC) and said single chain binding module (scBM) are separated by said linkers L1 and L2, thus forming a sequence from the N terminus to the C terminus selected from the group consisting of
[LC-L1-scBM-L2-LC],
[LC-L1-LC-L2-scBM] and
[scBM-L1-LC-L2-LC].

2. Bispecific binding molecule according to claim 1, wherein the sequence of elements from the N terminus to the C terminus in said single chain component is selected from the group consisting of
[LC-L1-scBM-L2-LC],
[LC-L1-LC-L2-scBM]

3. Bispecific binding molecule according to claim 1, wherein said first target is selected from the group consisting of amyloid-p peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), beta-secretase 1 (BACE1), superoxide dismutase (SOD), huntingtin, transthyretin, P-secretase 1, epidermal growth factor, epidermal growth factor receptor 2, Tau, phosphorylated Tau or fragments thereof, apolipoprotein E4, CD20, prion protein, leucine rich repeat kinase 2, parkin, presenilin 2, gamma secretase, death receptor 6, amyloid-p precursor protein, p75 neurotrophin receptor, neuregulin and caspase 6.

4. Bispecific binding molecule according to claim 1, wherein said monoclonal antibody with affinity for said first target is an anti-Ap antibody selected from the group consisting of lecanemab, gantenerumab, aducanumab, donanemab, PBD-006 and KHK6640.

5. Bispecific binding molecule according to claim 1, wherein said monoclonal antibody with affinity for said first target is the anti-alpha-synuclein antibody ABBV0805.

6. Bispecific binding molecule according to claim 1, wherein said scBM is of a type selected from the group consisting of scFv, 20 scFab, VHH and VNAR.

7. Bispecific binding molecule according to claim 6, wherein said scBM is a scFv.

8. Bispecific binding molecule according to claim 1, wherein said second target is selected from the group consisting of transferrin receptor 1 (TfR1), insulin receptor (InsR), insulin-like growth factor 1 receptor (IGF-1R), low density lipoprotein receptor-related protein 8 (Lrp8), low density lipoprotein receptor-related protein 1 (Lrp1), CD98, transmembrane protein 50A (TMEM50A), glucose transporter 1 (Glut1), basigin (BSG) and heparin-binding epidermal growth factor-like growth factor.

9. Bispecific binding molecule according to claim 1, wherein both of said amino acid linkers L1 and L2 are flexible linkers.

10. Bispecific binding molecule according to claim 9, wherein said flexible linkers comprise glycine, serine, alanine and/or threonine residues.

11. Bispecific binding molecule according to claim 1, wherein at least one of said amino acid linkers L1 and L2 is between 10 and 50 amino acid residues long, or between 10 and 30 amino acid residues long, or between 15 and 25 amino acid residues long or between 10 and 20 amino acids long.

12. Bispecific binding molecule according to claim 1, wherein said amino acid linkers L1 and L2 are of the same length, or wherein said amino acid linkers L1 and L2 are of different length.

13. Pharmaceutical composition, comprising a bispecific binding molecule according to claim 1 and a pharmaceutically acceptable carrier or excipient.

14. A method of therapeutic treatment, prophylactic treatment, diagnosis in vivo or prognosis in vivo comprising administering to a subject in need thereof a bispecific binding molecule according to claim 1.

15. The method according to claim 14, wherein said therapy, prophylaxis, in vivo diagnosis or in vivo prognosis is with respect to a neurodegenerative disorder, brain cancer, multiple sclerosis or a lysosomal storage disease.

16. The method of claim 15, wherein the neurodegenerative disorder is selected from the group consisting of Alzheimer's disease and other disorders associated with Aβ protein aggregation, traumatic brain injury (TBI), Lewy body dementia (LBD), Down's syndrome (DS), amyotrophic lateral sclerosis (ALS), frontotemporal dementia, tauopathy, systemic amyloidosis, atherosclerosis, Parkinson's disease (PD), Parkinson's disease dementia (PDD), the Lewy body variant of Alzheimer's disease, multiple system atrophy, psychosis, schizophrenia, Creutzfeldt-Jakob disease, Huntington's disease, familial amyloid neuropathy.
